SEQUENCE LISTING (25 sequences)

SEQ ID NO: 1 - DNA, 504 bp, Escherichia coli
atgttaaaaataaaatacttattaataggtctttcactgtcagctatgagttcatactcactagctgcagcggggcccactctaaccaaagaactggcattaaatgtgctttctcctgcagctctggatgcaacttgggctcctcaggataatttaacattatccaatactggcgtttctaatactttggtgggtgttttgactctttcaaataccagtattgatacagttagcattgcgagtacaagtgtttctgatacatctaagaatggtacagtaacttttgcacatgagacaaataactctgctagctttgccaccaccatttcaacagataatgccaacattacgttggataaaaatgctggaaatacgattgttaaaactacaaatgggagtcagttgccaactaatttaccacttaagtttattaccactgaaggtaacgaacatttagtttcaggtaattaccgtgcaaatataacaattacttcgacaattaaa

SEQ ID NO: 2 - protein, 168 aa, Escherichia coli
MLKIKYLLIGLSLSAMSSYSLAAAGPTLTKELALNVLSPAALDATWAPQDNLTLSNTGVSNTLVGVLTLSNTSIDTVSIASTSVSDTSKNGTVTFAHETNNSASFATTISTDNANITLDKNAGNTIVKTTNGSQLPTNLPLKFITTEGNEHLVSGNYRANITITSTIK

SEQ ID NO: 3 - DNA, 372 bp, Escherichia coli
atgaataaagtaaaattttatgttttatttacggcgttactatcctctctatgtgcatacggagctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaac

SEQ ID NO: 4 - protein, 124 aa, Escherichia coli
MNKVKFYVLFTALLSSLCAYGAPQSITELCSEYRNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 5 - DNA, 57 bp, Escherichia coli
aatagtagcaattactgctgtgaattgtgttgtaatccgctctgtaccgggtgctat

SEQ ID NO: 6 - protein, 19 aa, Escherichia coli
NSSNYCCELCCNPLCTGCY

SEQ ID NO: 7 - DNA, 66 bp, Escherichia coli
atgttaaaaataaaatacttattaataggtctttcactgtcagctatgagttcatactcactagct

SEQ ID NO: 8 - protein, 22 aa, Escherichia coli
MLKIKYLLIGLSLSAMSSYSLA

SEQ ID NO: 9 - DNA, 28 bp, Escherichia coli
ttacctaggggtacccagcttttgttcc

SEQ ID NO: 10 - DNA, 108 bp, Escherichia coli
acttcctaggctagtctagattaatagcacccggtacagagcccattacaacacaattcacagcagtaattgctactattcccggggttttccatactgattgccgca

SEQ ID NO: 11 - DNA, 453 bp, Artificial Sequence
  CS3 (export signal)LTbST fusion
atgttaaaaataaaatacttattaataggtctttcactgtcagctatgagttcatactcactagctgcagcggggcccgctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaaccccgggaatagtagcaattactgctgtgaattgtgttgtaatccgctctgtaccgggtgctattaa

SEQ ID NO: 12 - protein, 150 aa, Artificial Sequence
  CS3 (export signal)LTbST fusion
MLKIKYLLIGLSLSAMSSYSLAAAGPAPQSITELCSEYRNTQIYTINDKILSYTESMAGKREMVIITFKSGATFQVEVPGSQHIDSQKKAIERMKDTLRITYLTETKIDKLCVWNNKTPNSIAAISMENPGNSSNYCCELCCNPLCTGCY

SEQ ID NO: 13 - DNA, 885 bp, Artificial Sequence
  CS3 (full length)LTbST fusion
atgttaaaaataaaatacttattaataggtctttcactgtcagctatgagttcatactcactagctgcagcggggcccactctaaccaaagaactggcattaaatgtgctttctcctgcagctctggatgcaacttgggctcctcaggataatttaacattatccaatactggcgtttctaatactttggtgggtgttttgactctttcaaataccagtattgatacagttagcattgcgagtacaagtgtttctgatacatctaagaatggtacagtaacttttgcacatgagacaaataactctgctagctttgccaccaccatttcaacagataatgccaacattacgttggataaaaatgctggaaatacgattgttaaaactacaaatgggagtcagttgccaactaatttaccacttaagtttattaccactgaaggtaacgaacatttagtttcaggtaattaccgtgcaaatataacaattacttcgacaattaaacccggggctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaaccccgggaatagtagcaattactgctgtgaattgtgttgtaatccgctctgtaccgggtgctattaa

SEQ ID NO: 14 - protein, 294 aa, Artificial Sequence
  CS3 (full length)LTbST fusion
MLKIKYLLIGLSLSAMSSYSLAAAGPTLTKELALNVLSPAALDATWAPQDNLTLSNTGVSNTLVGVLTLSNTSIDTVSIASTSVSDTSKNGTVTFAHETNNSASFATTISTDNANITLDKNAGNTIVKTTNGSQLPTNLPLKFITTEGNEHLVSGNYRANITITSTIKPGAPQSITELCSEYRNTQIYTINDKILSYTESMAGKREMVIITFKSGATFQVEVPGSQHIDSQKKAIERMKDTLRITYLTETKIDKLCVWNNKTPNSIAAISMENPGNSSNYCCELCCNPLCTGCY

SEQ ID NO: 15 - DNA, 474 bp, Salmonella sp.
ctcgagattgccatcgcggatgtcgcctgtcttatctaccatcataaacatcatttgcctatggctcacgacagtataggcaatgccgttttttatattgctaattgtttcgccaatcaacgcaaaagtatggcgattgctaaagccgtctccctgggcggtagattagccttaaccgcgacggtaatgactcattcatactggagtggtagtttgggactacagcctcatttattagagcgtcttaatgatattacctatggactaatgagttttactcgcttcggtatggatgggatggcaatgaccggtatgcaggtcagcagcccattatatcgtttgctggctcaggtaacgccagaacaacgtgcgccggagtaatcgttttcaggtatataccggatgttcattgctttctaaattttgctatgttgccagtatccttacgatgtatttattttaaggaaaagccat

SEQ ID NO: 16 - DNA, 72 bp, Artificial Sequence
  CS3 export signal sequence plus linker 1
atgttaaaaataaaatacttattaataggtctttcactgtcagctatgagttcatactcactagctgcagcg

SEQ ID NO: 17 - protein, 24 aa, Artificial Sequence
  CS3 export signal sequence plus linker 1
MLKIKYLLIGLSLSAMSSYSLAAA

SEQ ID NO: 18 - DNA, 78 bp, Artificial Sequence
  CS3 export signal sequence plus linker 2
atgttaaaaataaaatacttattaataggtctttcactgtcagctatgagttcatactcactagctgcagcggggccc

SEQ ID NO: 19 - protein, 26 aa, Artificial Sequence
  CS3 export signal sequence plus linker 2
MLKIKYLLIGLSLSAMSSYSLAAAGP

SEQ ID NO: 20 - protein, 4 aa, Artificial Sequence
  Amino acid linker
AAPG

SEQ ID NO: 21 - protein, 4 aa, Artificial Sequence
  Amino acid linker
AAGP

SEQ ID NO: 22 - DNA, 2638 bp, Artificial Sequence
  ssaG promoter, full length CS3, C diff toxin B C terminal repeat domain
ctcgagattgccatcgcggatgtcgcctgtcttatctaccatcataaacatcatttgcctatggctcacgacagtataggcaatgccgttttttatattgctaattgtttcgccaatcaacgcaaaagtatggcgattgctaaagccgtctccctgggcggtagattagccttaaccgcgacggtaatgactcattcatactggagtggtagtttgggactacagcctcatttattagagcgtcttaatgatattacctatggactaatgagttttactcgcttcggtatggatgggatggcaatgaccggtatgcaggtcagcagcccattatatcgtttgctggctcaggtaacgccagaacaacgtgcgccggagtaatcgttttcaggtatataccggatgttcattgctttctaaattttgctatgttgccagtatccttacgatgtatttattttaaggaaaagccatatgttaaaaataaaatacttattaataggtctttcactgtcagctatgagttcatactcactagctgcagcggggcccactctaaccaaagaactggcattaaatgtgctttctcctgcagctctggatgcaacttgggctcctcaggataatttaacattatccaatactggcgtttctaatactttggtgggtgttttgactctttcaaataccagtattgatacagttagcattgcgagtacaagtgtttctgatacatctaagaatggtacagtaacttttgcacatgagacaaataactctgctagctttgccaccaccatttcaacagataatgccaacattacgttggataaaaatgctggaaatacgattgttaaaactacaaatgggagtcagttgccaactaatttaccacttaagtttattaccactgaaggtaacgaacatttagtttcaggtaattaccgtgcaaatataacaattacttcgacaattaaacccgggaagttttatatcaacaacttcggcatgatggtgtctggcttgatctacatcaacgatagcctctattatttcaagccgcccgttaataacttaatcacaggcttcgtgacagtaggtgatgacaaatactattttaatccgatcaatggaggcgcagcaagtattggtgaaacgataatcgacgacaagaactattattttaaccaatcaggagtgctgcaaactggtgtgttttccaccgaggacggctttaagtacttcgcccccgcgaacaccctggacgaaaaccttgagggtgaagccattgacttcactggtaaacttattatcgacgaaaacatctactattttgatgataactacagaggcgcagtggagtggaaagagctggacggggaaatgcattacttttccccagagacaggtaaagctttcaaaggtctgaatcagattggggattacaaatattacttcaactctgacggtgtcatgcagaagggatttgtgtcaatcaacgataataagcactactttgatgactcaggagtaatgaaggtgggctacacggagattgacggaaaacatttctatttcgccgaaaatggtgaaatgcagattggcgttttcaataccgaggatggcttcaagtattttgctcatcacaatgaggatctgggaaacgaagaaggcgaggaaatttcctactcgggcatactgaattttaacaataaaatatattatttcgacgacagttttacggcggttgttgggtggaaggatttagaagatggtagtaaatactacttcgatgaggacacggccgaagcctatatcggtttgtcgctgattaatgatggacagtactattttaatgacgacggcattatgcaagttgggttcgtgaccattaacgacaaagtgttttatttttcagactcaggaattatcgagagcggggttcaaaacattgatgataattatttttacatagacgataatgggatcgttcagatcggggtgttcgacacatctgacggttacaaatattttgctcccgcaaatacggtgaacgacaacatttacgggcaggcagtggaatattcgggtttggttagagttggcgaggatgtctactattttggcgagacatacacgattgaaacggggtggatttacgatatggagaacgaaagcgataaatattactttaacccagaaacaaagaaggcctgcaaaggtatcaatttaatcgatgatatcaaatactatttcgacgaaaagggtatcatgcgtactgggctgatcagctttgagaacaataattactatttcaatgaaaatggggaaatgcaatttggatatattaatatagaagataagatgttttatttcggggaggatggtgtgatgcagatcggcgttttcaacaccccggacgggtttaaatatttcgcacatcagaatacactggatgagaacttcgagggtgagtctattaactacaccgggtggctggacttagacgagaaacgctactatttcacagacgagtacattgcagctactggttcggtcatcattgatggcgaggaatattatttcgacccggataccgcccagttagtgatctccgagtaatctagactagcctagg

SEQ ID NO: 23 - DNA, 2206 bp, Artificial Sequence
  ssaG promoter, CS3 signal peptide, C diff toxin B C terminal repeat domain
ctcgagattgccatcgcggatgtcgcctgtcttatctaccatcataaacatcatttgcctatggctcacgacagtataggcaatgccgttttttatattgctaattgtttcgccaatcaacgcaaaagtatggcgattgctaaagccgtctccctgggcggtagattagccttaaccgcgacggtaatgactcattcatactggagtggtagtttgggactacagcctcatttattagagcgtcttaatgatattacctatggactaatgagttttactcgcttcggtatggatgggatggcaatgaccggtatgcaggtcagcagcccattatatcgtttgctggctcaggtaacgccagaacaacgtgcgccggagtaatcgttttcaggtatataccggatgttcattgctttctaaattttgctatgttgccagtatccttacgatgtatttattttaaggaaaagccatatgttaaaaataaaatacttattaataggtctttcactgtcagctatgagttcatactcactagctgcagcggggcccaagttttatatcaacaacttcggcatgatggtgtctggcttgatctacatcaacgatagcctctattatttcaagccgcccgttaataacttaatcacaggcttcgtgacagtaggtgatgacaaatactattttaatccgatcaatggaggcgcagcaagtattggtgaaacgataatcgacgacaagaactattattttaaccaatcaggagtgctgcaaactggtgtgttttccaccgaggacggctttaagtacttcgcccccgcgaacaccctggacgaaaaccttgagggtgaagccattgacttcactggtaaacttattatcgacgaaaacatctactattttgatgataactacagaggcgcagtggagtggaaagagctggacggggaaatgcattacttttccccagagacaggtaaagctttcaaaggtctgaatcagattggggattacaaatattacttcaactctgacggtgtcatgcagaagggatttgtgtcaatcaacgataataagcactactttgatgactcaggagtaatgaaggtgggctacacggagattgacggaaaacatttctatttcgccgaaaatggtgaaatgcagattggcgttttcaataccgaggatggcttcaagtattttgctcatcacaatgaggatctgggaaacgaagaaggcgaggaaatttcctactcgggcatactgaattttaacaataaaatatattatttcgacgacagttttacggcggttgttgggtggaaggatttagaagatggtagtaaatactacttcgatgaggacacggccgaagcctatatcggtttgtcgctgattaatgatggacagtactattttaatgacgacggcattatgcaagttgggttcgtgaccattaacgacaaagtgttttatttttcagactcaggaattatcgagagcggggttcaaaacattgatgataattatttttacatagacgataatgggatcgttcagatcggggtgttcgacacatctgacggttacaaatattttgctcccgcaaatacggtgaacgacaacatttacgggcaggcagtggaatattcgggtttggttagagttggcgaggatgtctactattttggcgagacatacacgattgaaacggggtggatttacgatatggagaacgaaagcgataaatattactttaacccagaaacaaagaaggcctgcaaaggtatcaatttaatcgatgatatcaaatactatttcgacgaaaagggtatcatgcgtactgggctgatcagctttgagaacaataattactatttcaatgaaaatggggaaatgcaatttggatatattaatatagaagataagatgttttatttcggggaggatggtgtgatgcagatcggcgttttcaacaccccggacgggtttaaatatttcgcacatcagaatacactggatgagaacttcgagggtgagtctattaactacaccgggtggctggacttagacgagaaacgctactatttcacagacgagtacattgcagctactggttcggtcatcattgatggcgaggaatattatttcgacccggataccgcccagttagtgatctccgagtaatctagactagcctagg

SEQ ID NO: 24 - DNA, 3673 bp, Artificial Sequence
  ssaG promoter, full length CS3, C diff toxin A C terminal repeat domain
ctcgagattgccatcgcggatgtcgcctgtcttatctaccatcataaacatcatttgcctatggctcacgacagtataggcaatgccgttttttatattgctaattgtttcgccaatcaacgcaaaagtatggcgattgctaaagccgtctccctgggcggtagattagccttaaccgcgacggtaatgactcattcatactggagtggtagtttgggactacagcctcatttattagagcgtcttaatgatattacctatggactaatgagttttactcgcttcggtatggatgggatggcaatgaccggtatgcaggtcagcagcccattatatcgtttgctggctcaggtaacgccagaacaacgtgcgccggagtaatcgttttcaggtatataccggatgttcattgctttctaaattttgctatgttgccagtatccttacgatgtatttattttaaggaaaagccatatgttaaaaataaaatacttattaataggtctttcactgtcagctatgagttcatactcactagctgcagcggggcccactctaaccaaagaactggcattaaatgtgctttctcctgcagctctggatgcaacttgggctcctcaggataatttaacattatccaatactggcgtttctaatactttggtgggtgttttgactctttcaaataccagtattgatacagttagcattgcgagtacaagtgtttctgatacatctaagaatggtacagtaacttttgcacatgagacaaataactctgctagctttgccaccaccatttcaacagataatgccaacattacgttggataaaaatgctggaaatacgattgttaaaactacaaatgggagtcagttgccaactaatttaccacttaagtttattaccactgaaggtaacgaacatttagtttcaggtaattaccgtgcaaatataacaattacttcgacaattaaacccgggacatattactacgacgaagattcgaagttggtcaagggcctgataaacataaacaactcgttattttatttcgatcctattgaatttaacctggtgacggggtggcagaccataaacgggaagaagtactactttgacatcaataccggcgcagcattgatttcatataagataattaacggcaagcatttctactttaacaacgatggagtcatgcaactgggagtctttaagggtcccgacggcttcgaatactttgccccagcgaacacccaaaacaacaatattgaggggcaggcgattgtctatcaatcaaagtttttgacgctgaacggtaagaaatactattttgataacgattcgaaagcagtcacggggtggcggattattaacaacgaaaaatattattttaatccaaataatgctatcgcagcagtcgggcttcaagtgatcgataataataagtactacttcaatccagatacggctattatttcaaaagggtggcagactgtcaacggctccaggtattatttcgacactgatactgctatcgctttcaacgggtataagacaatcgatggtaagcatttctactttgatagcgactgcgtggttaaaattggtgtattcagtacctctaatggatttgagtacttcgctcctgcaaacacttacaataacaatattgaaggtcaggccatcgtataccaaagcaagttcctcaccttaaatggcaaaaagtactatttcgacaacaatagcaaagcggtcaccggttggcagaccattgatagtaaaaaatattattttaataccaacactgcggaagctgctaccggatggcagacaatcgacggcaagaagtattatttcaacaccaatacagcagaagcggccacagggtggcaaacgatcgacgggaagaagtactactttaatactaacacggccattgctagcaccggttataccattattaatgggaaacacttttacttcaacactgacggcattatgcagatcggtgtattcaaagggcctaacggcttcgaatatttcgcaccggccaatacagacgcgaacaatatagaaggacaggcgattctgtatcagaatgaattcctgaccctgaatggtaagaaatattacttcggcagcgattctaaggccgtcaccgggtggcggataatcaataataaaaagtactatttcaacccgaataacgcgattgcagctattcacctgtgcacgatcaacaatgataagtattattttagctatgatgggatccttcaaaatggatatattacaatagaaagaaataacttctatttcgatgcgaataatgagtctaaaatggtgactggcgttttcaaaggcccaaatgggttcgaatacttcgctccggcgaacacacacaacaacaatattgaagggcaggcaatagtgtatcagaataaattcttgacgctgaatggtaaaaagtactactttgataatgattcgaaagcggtaacaggctggcagaccatagacggcaagaaatattactttaatctgaatactgccgaagctgcgacgggctggcaaaccatagacggaaagaaatattattttaatctgaacaccgcagaggccgccaccggatggcagaccatcgacgggaagaaatactatttcaacactaataccttcatagcgagtacggggtatacctcgatcaatggcaagcatttctactttaacaccgacgggattatgcagatcggtgttttcaaggggccgaacggcttcgaatacttcgctcccgcaaacacacacaacaacaacatcgagggacaggctatactgtatcaaaataaatttcttacgttaaatggcaagaagtattattttgggtcggacagcaaagcagtgaccggtttgcgtaccatagatggtaagaaatattattttaatactaacacggcagtagccgttaccggatggcagactattaatgggaagaaatactattttaacactaacacgagcattgcctcgactggctacacgatcattagcgggaaacacttctacttcaacacggatggtattatgcagataggtgtctttaaaggtcctgacggttttgagtacttcgcacccgccaacaccgacgctaataacatagaggggcaagctatcaggtatcagaatcgcttcctttacctgcatgataacatctattacttcgggaacaacagtaaggctgctaccgggtgggtgacaattgacggtaatcgctattatttcgagcctaacacagcaatgggagccaatggctataagactatcgataacaaaaatttttactttcggaacggtttgcctcaaatcggggtttttaaaggatctaacggcttcgagtactttgccccggcgaacacggatgccaacaatattgagggccaggcgataaggtaccagaaccgctttctgcatctcttgggtaaaatctattacttcggcaacaactcaaaggcggtaacaggatggcaaactataaacgggaaggtttactattttatgcctgatacggccatggctgcggcgggaggcctgttcgaaattgacggtgttatatactttttcggtgtggacggtgttaaggccccaggcatttactaatctagactagcctagg

SEQ ID NO: 25 - DNA, 3241 bp, Artificial Sequence
  ssaG promoter, CS3 signal peptide, C diff toxin A C terminal repeat domain
ctcgagattgccatcgcggatgtcgcctgtcttatctaccatcataaacatcatttgcctatggctcacgacagtataggcaatgccgttttttatattgctaattgtttcgccaatcaacgcaaaagtatggcgattgctaaagccgtctccctgggcggtagattagccttaaccgcgacggtaatgactcattcatactggagtggtagtttgggactacagcctcatttattagagcgtcttaatgatattacctatggactaatgagttttactcgcttcggtatggatgggatggcaatgaccggtatgcaggtcagcagcccattatatcgtttgctggctcaggtaacgccagaacaacgtgcgccggagtaatcgttttcaggtatataccggatgttcattgctttctaaattttgctatgttgccagtatccttacgatgtatttattttaaggaaaagccatatgttaaaaataaaatacttattaataggtctttcactgtcagctatgagttcatactcactagctgcagcggggcccacatattactacgacgaagattcgaagttggtcaagggcctgataaacataaacaactcgttattttatttcgatcctattgaatttaacctggtgacggggtggcagaccataaacgggaagaagtactactttgacatcaataccggcgcagcattgatttcatataagataattaacggcaagcatttctactttaacaacgatggagtcatgcaactgggagtctttaagggtcccgacggcttcgaatactttgccccagcgaacacccaaaacaacaatattgaggggcaggcgattgtctatcaatcaaagtttttgacgctgaacggtaagaaatactattttgataacgattcgaaagcagtcacggggtggcggattattaacaacgaaaaatattattttaatccaaataatgctatcgcagcagtcgggcttcaagtgatcgataataataagtactacttcaatccagatacggctattatttcaaaagggtggcagactgtcaacggctccaggtattatttcgacactgatactgctatcgctttcaacgggtataagacaatcgatggtaagcatttctactttgatagcgactgcgtggttaaaattggtgtattcagtacctctaatggatttgagtacttcgctcctgcaaacacttacaataacaatattgaaggtcaggccatcgtataccaaagcaagttcctcaccttaaatggcaaaaagtactatttcgacaacaatagcaaagcggtcaccggttggcagaccattgatagtaaaaaatattattttaataccaacactgcggaagctgctaccggatggcagacaatcgacggcaagaagtattatttcaacaccaatacagcagaagcggccacagggtggcaaacgatcgacgggaagaagtactactttaatactaacacggccattgctagcaccggttataccattattaatgggaaacacttttacttcaacactgacggcattatgcagatcggtgtattcaaagggcctaacggcttcgaatatttcgcaccggccaatacagacgcgaacaatatagaaggacaggcgattctgtatcagaatgaattcctgaccctgaatggtaagaaatattacttcggcagcgattctaaggccgtcaccgggtggcggataatcaataataaaaagtactatttcaacccgaataacgcgattgcagctattcacctgtgcacgatcaacaatgataagtattattttagctatgatgggatccttcaaaatggatatattacaatagaaagaaataacttctatttcgatgcgaataatgagtctaaaatggtgactggcgttttcaaaggcccaaatgggttcgaatacttcgctccggcgaacacacacaacaacaatattgaagggcaggcaatagtgtatcagaataaattcttgacgctgaatggtaaaaagtactactttgataatgattcgaaagcggtaacaggctggcagaccatagacggcaagaaatattactttaatctgaatactgccgaagctgcgacgggctggcaaaccatagacggaaagaaatattattttaatctgaacaccgcagaggccgccaccggatggcagaccatcgacgggaagaaatactatttcaacactaataccttcatagcgagtacggggtatacctcgatcaatggcaagcatttctactttaacaccgacgggattatgcagatcggtgttttcaaggggccgaacggcttcgaatacttcgctcccgcaaacacacacaacaacaacatcgagggacaggctatactgtatcaaaataaatttcttacgttaaatggcaagaagtattattttgggtcggacagcaaagcagtgaccggtttgcgtaccatagatggtaagaaatattattttaatactaacacggcagtagccgttaccggatggcagactattaatgggaagaaatactattttaacactaacacgagcattgcctcgactggctacacgatcattagcgggaaacacttctacttcaacacggatggtattatgcagataggtgtctttaaaggtcctgacggttttgagtacttcgcacccgccaacaccgacgctaataacatagaggggcaagctatcaggtatcagaatcgcttcctttacctgcatgataacatctattacttcgggaacaacagtaaggctgctaccgggtgggtgacaattgacggtaatcgctattatttcgagcctaacacagcaatgggagccaatggctataagactatcgataacaaaaatttttactttcggaacggtttgcctcaaatcggggtttttaaaggatctaacggcttcgagtactttgccccggcgaacacggatgccaacaatattgagggccaggcgataaggtaccagaaccgctttctgcatctcttgggtaaaatctattacttcggcaacaactcaaaggcggtaacaggatggcaaactataaacgggaaggtttactattttatgcctgatacggccatggctgcggcgggaggcctgttcgaaattgacggtgttatatactttttcggtgtggacggtgttaaggccccaggcatttactaatctagactagcctagg

(19) United States
(12) Patent Application Publication
(10) Pub. No.: US 2012/0064114 A1
(43) Pub. Date: Mar. 15, 2012

(54) USE OF E. COLI SURFACE ANTIGEN 3 SEQUENCES FOR THE EXPORT OF HETEROLOGOUS ANTIGENS

(75) Inventors: Jonathan Telfer, Reading (GB); Mark Richard Redfern, Reading (GB)

(73) Assignee: Emergent Product Development United Kingdom, Wokingham, Berkshire (GB)

(21) Appl. No.: 13/125,533

(22) PCT Filed: Oct. 21, 2009
(86) PCT No.: PCT/US09/61528

§ 371 Date: Nov. 22, 2011

Related U.S. Application Data
(60) Provisional application No. 61/107,113, filed on Oct. 21, 2008.

(51) Int. Cl. A61K 35/74; C12N 1/21; C12P 21/02; A61P 31/04; C12P 21/00; C12N 15/74; A61P 37/04

(52) U.S. Cl. 424/200.1; 435/320.1; 435/252.3; 435/69.3

(57) ABSTRACT

The present invention provides an export signal system based on E. coli CS3 antigen for directing the secretion of foreign antigens from host cells. In particular, the invention describes genetic constructs encoding fusion proteins that contain a CS3 export signal fused to at least one heterologous amino acid sequence. Attenuated microorganisms expressing the fusion proteins and pharmaceutical compositions comprising such attenuated microorganisms are also disclosed.

### CROSS-REFERENCE TO RELATED APPLICATIONS

This application claims the benefit of priority of U.S. Provisional Application No. 61/107,113, filed Oct. 21, 2008, which is herein incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: EMER 005_{—}01WO_SeqList_ST25.txt, date recorded: Oct. 21, 2009, file size 30 kilobytes).

### FIELD OF THE INVENTION

The present invention relates to the fields of medicine, immunology, and vaccine development. The invention describes the use of signal peptides derived from an E. coli colonization factor protein to export heterologous antigens from host cells. Constructs, microorganisms comprising the constructs, and vaccines produced from such microorganisms are also disclosed.

### BACKGROUND OF THE INVENTION

Use of live attenuated microorganisms to deliver antigens to the immune system has proven to be a very effective strategy for inducing a protective immune response against a number of pathogens. Several live attenuated vaccines have been developed and safely used for a number of infections and diseases, including tuberculosis, typhoid, salmonellosis, polio, measles, mumps, and cholera. Immune responses elicited by these live attenuated vaccines are typically stronger and longer in duration than those induced by subunit or inactivated vaccines. One reason for the strong immune response to attenuated microorganisms may be that these microorganisms continually present antigen to the immune system in a similar manner to that which would occur with a natural infection. In addition, the microorganisms stimulate a cell-mediated immune response since they are capable of being phagocytosed by macrophages, which in turn break down the microorganisms and present antigens of the microorganisms on their surface.

Live attenuated microorganisms, such as live attenuated bacteria, have also been used as vaccine platforms for other pathogens with various levels of success. See, for instance, Parida, S K et al., Ann. N. Y. Acad. Sci., Vol. 1056:366-78 (2005) and Khan, S A et al., Vaccine, Vol. 21(5-6):538-548 (2003). These attenuated bacteria must be able to express one or more heterologous antigens of a pathogen (or pathogens) to elicit an effective immune response. In order to elicit a humoral immune response, it is desirable that the live attenuated bacterial vaccine platform be able to express the heterologous antigen(s) either on the cell surface or be able to secrete the expressed protein from the cell. However, structural changes in the protein during the expression and export process can make the generation of an effective immune response challenging. Thus, development of proteins and/or peptides that can act as fusion partners for a variety of foreign antigens and can be adapted for expression in attenuated microorganisms, such as attenuated bacteria, is desirable.

### SUMMARY OF THE INVENTION

The present invention provides a novel use for coli surface antigen 3 (CS3) protein and fragments thereof for exporting foreign antigens to the outer surface of host cells. The invention is based, in part, on the discovery that the export signal sequence from E. coli CS3 protein can be incorporated into fusion proteins to direct the cell surface expression of a heterologous antigen by a host cell (e.g., Salmonella cell). This technique allows for antigen epitopes of almost any size to be effectively displayed on the outer surface of a host cell. Accordingly, the present invention provides genetic constructs encoding fusion proteins comprising the CS3 export signal sequence and methods of using the constructs to express foreign antigens on the surface of host cells.

In one embodiment, the genetic construct comprises a promoter operably linked to a nucleic acid encoding a fusion protein, wherein said fusion protein comprises an amino acid sequence from E. coli CS3 protein consisting essentially of an export signal fused to at least one heterologous antigen amino acid sequence. The promoter may be an inducible promoter that may be induced in vivo, such as the Salmonella ssaG promoter. In some embodiments, the genetic construct may further comprise a linker amino acid sequence positioned between the CS3 export amino acid sequence and the heterologous antigen amino acid sequence, for example to facilitate the proper folding of the heterologous antigen. In one embodiment, the linker may be comprised of proline, glycine, alanine, serine, threonine, and/or asparagine residues.

The present invention also encompasses a method for displaying at least one heterologous antigen on a cell. In one embodiment, the method comprises expressing in the cell one or more genetic construct(s) encoding one or more fusion protein(s), wherein said fusion protein comprises an amino acid sequence from E. coli CS3 protein consisting essentially of an export signal fused to at least one heterologous antigen amino acid sequence. The fusion protein may be secreted from the cell following delivery of the genetic construct. In some embodiments, the cell may be a bacterial cell. In a preferred embodiment, the bacterial cell is an attenuated Salmonella. The attenuated Salmonella may comprise one or more deletions or inactivations in genes involved in the biosynthesis of aromatic compounds and/or genes located in the Salmonella pathogenicity island 2 (SPI-2).

The present invention also provides attenuated microorganisms comprising the genetic constructs disclosed herein. In one embodiment, the attenuated microorganism expresses the fusion protein encoded by the genetic construct. In another embodiment, the attenuated microorganism expresses the heterologous antigen encoded by the genetic construct on its outer surface. For instance, in one embodiment of the invention, the microorganism uses CS3 signal sequence to deliver the heterologous antigen to the cell surface. In another embodiment, the attenuated microorganism secretes the fusion protein encoded by the genetic construct.

In some embodiments the attenuated microorganism is an attenuated Gram negative bacterium. In one embodiment, the attenuated Gram negative bacterium is selected from the group consisting of Salmonella, Escherichia coli, Vibrio and Shigella. In some embodiments of the invention, the attenuated microorganism is an attenuated Salmonella spp., such as an attenuated Salmonella enterica serovar (e.g., Salmonella enterica serovar Typhi or Salmonella enterica serovar Typhimurium). The attenuated Salmonella spp. may comprise one or more inactivating mutations in genes encoded on a SPI-2 region. In one embodiment, the attenuated Salmonella further comprises one or more inactivating mutations in genes required for the biosynthesis of aromatic compounds. In another embodiment, the attenuated Salmonella has a deletion or inactivation of the ssaV gene and the aroC gene. In another embodiment, the attenuated Salmonella has a deletion or inactivation of the ssaJ gene and the aroC gene.

The present invention also encompasses vaccine compositions comprising the attenuated microorganisms disclosed herein and a pharmaceutically acceptable carrier. In one embodiment, the vaccine composition comprises an attenuated Salmonella comprising a genetic construct encoding a fusion protein under the control of an inducible promoter, wherein said fusion protein comprises an export signal sequence from an E. coli CS3 protein and an amino acid sequence from at least one heterologous antigen. In another embodiment, the heterologous antigen is heat labile enterotoxin or heat stable enterotoxin from enterotoxigenic E. coli (ETEC) or a fusion between the two. In some embodiments, the vaccine composition may further comprise an adjuvant.

The present invention also provides a method for immunizing a subject against a pathogen. In one embodiment, the method comprises administering a vaccine composition comprising an attenuated microorganism to the subject, wherein the attenuated microorganism expresses a fusion protein, said fusion protein comprising an amino acid sequence from E. coli CS3 protein consisting essentially of an export signal fused to at least one heterologous antigen amino acid sequence from the pathogen. In one embodiment of the invention, the attenuated microorganism capable of expressing a heterologous antigen targets the heterologous antigen to the cell surface by way of an E. coli CS3 export signal sequence. The vaccine composition may be administered orally to the subject. In some embodiments, the vaccine composition induces mucosal immunity against the pathogen in the subject. In one embodiment, the pathogen is enterotoxigenic E. coli (ETEC).

The present invention provides methods of treating or preventing an infection caused by a pathogen in a subject. In one embodiment, the method comprises administering to a subject an attenuated microorganism comprising a nucleic acid encoding a CS3 signal peptide and an antigen of the pathogen. The infection may be a bacterial, viral, fungal, or parasitic infection.

In another embodiment, the present invention provides a method of treating cancer in a subject comprising administering to the subject an attenuated microorganism comprising a nucleic acid encoding a CS3 signal peptide and a cancer antigen. Several types of cancer, including breast, ovarian, colorectal, prostate, lung, liver, skin, and pancreatic cancer, may be treated with the methods of the invention. In another embodiment, the invention provides a method of delivering a therapeutic protein, such as a cytokine, to treat cancer or another disorder or disease in a subject or to boost the immune response of a subject to the administered pharmaceutical composition. The method may comprise administering to the subject an attenuated microorganism comprising a nucleic acid encoding a CS3 signal peptide and a cytokine.

The present invention also encompasses isolated and purified recombinant fusion proteins encoded by the genetic constructs of the invention. In one embodiment, the recombinant fusion protein comprises an amino acid sequence from E. coli CS3 protein and an amino acid sequence from at least one heterologous antigen. The invention also includes isolated and purified antibodies to recombinant fusion proteins of the invention as well as isolated and purified antibodies to attenuated microorganisms (e.g. attenuated Salmonella) expressing the recombinant fusion proteins on their surface. Methods of generating antibodies in a subject, such as a human subject, using the recombinant fusion proteins and attenuated microorganisms of the invention are also included.

### BRIEF DESCRIPTION OF THE DRAWINGS

FIG. 1 shows the nucleotide (A; SEQ ID NO: 1) and amino acid (B; SEQ ID NO: 2) sequences for enterotoxigenic E. coli CS3 protein. The natural restriction site for ApaI is shown in bolded capital letters in the CS3 nucleotide sequence. The export signal sequence in the CS3 protein is underlined in both the nucleotide and amino acid sequences.

FIG. 2 shows the nucleotide and amino acid sequences for enterotoxigenic E. coli (ETEC) antigens. A. Nucleotide sequence for ETEC heat labile toxin B subunit (SEQ ID NO: 3). B. Amino acid sequence for ETEC heat labile toxin B subunit (SEQ ID NO: 4). The underlined region in both sequences illustrates the secretion signal found in the labile toxin sequence. C. Nucleotide sequence for detoxified ETEC stable toxin (SEQ ID NO: 5). D. Amino acid sequence for detoxified ETEC stable toxin (SEQ ID NO: 6). The ETEC stable toxin is detoxified by a single amino acid change from alanine to leucine at position 14.

FIG. 3 shows the vector map containing the nucleotide sequence (SEQ ID NO: 22) of the ssaG promoter, full length CS3, C. difficile toxin B construct.

FIG. 4 shows the vector map containing the nucleotide sequence (SEQ ID NO: 23) of the ssaG promoter, CS3 signal peptide, C. difficile toxin B construct.

FIG. 5 shows the vector map containing the nucleotide sequence (SEQ ID NO: 24) of the ssaG promoter, full length CS3, C. difficile toxin A construct.

FIG. 6 shows the vector map containing the nucleotide sequence (SEQ ID NO: 25) of the ssaG promoter, CS3 signal peptide, C. difficile toxin A construct.

### DETAILED DESCRIPTION OF THE INVENTION

The present invention is based, in part, on the discovery that the coli surface antigen 3 (CS3) protein from enterotoxigenic E. coli or fragments thereof, can be used to facilitate the cell surface expression or secretion of foreign antigens from a non-E.coli host cell (e.g., Salmonella cell). In enterotoxigenic E. coli, full length CS3 protein forms fimbriae, which extend from the bacterial cell surface and facilitate the attachment of the bacteria to the intestinal epithelium. Fusion proteins comprising CS3 or fragments thereof are targeted to the outer surface of host cells, where they are effectively presented to the immune system and induce an immune response.

The present invention provides genetic constructs encoding fusion proteins comprising CS3 protein or fragments thereof and a heterologous antigen. In one embodiment, the present invention provides a genetic construct comprising a promoter operably linked to a nucleic acid encoding a fusion protein, wherein said fusion protein comprises an amino acid sequence from E. coli CS3 protein consisting essentially of an export signal fused to at least one heterologous antigen amino acid sequence. As used herein, “export signal” refers to an amino acid sequence that is necessary and sufficient to target a protein containing that amino acid sequence to the outer surface of a host cell or direct the secretion of a protein containing that amino acid sequence from a host cell.

The fusion protein generally comprises an amino acid sequence from the E. coli CS3 protein and an amino acid sequence from at least one heterologous antigen. For instance, in one embodiment, the genetic construct comprises a nucleic acid encoding a CS3 polypeptide of SEQ ID NO: 2, 8, 17 or 19 or a polypeptide with at least about 85%, 90% or 95% identity to SEQ ID NO: 2, 8, 17 or 19. In one embodiment, the CS3 sequence is fused to the amino terminus of the heterologous antigen sequence.

In one embodiment, the E. coli CS3 protein is from enterotoxigenic E. coli (ETEC). In some embodiments, the amino acid sequence from an ETEC CS3 protein is SEQ ID NO: 8. In other embodiments, the amino acid sequence from an ETEC CS3 protein comprises SEQ ID NO: 8. In another embodiment, the amino acid sequence from the CS3 protein consists essentially of an export signal. By “consists essentially of an export signal,” it is meant that the CS3 polypeptide may contain a small number of additional amino acids or lack a small number of amino acids as compared to the CS3 polypeptide sequence of SEQ ID NO: 8. For instance, the invention includes a CS3 polypeptide corresponding to SEQ ID NOs: 17 or 19.

The amino acid sequence from the CS3 protein comprising an export signal may be from about 20, 21, 22, 23, 24, 25, 26, 27, 28, 29, 30, 35, 40, 45, 50, 55, or 60 amino acids in length. In another embodiment, the fusion protein may comprise an amino acid sequence of a full length E. coli CS3 protein such as that of SEQ ID NO: 2. In still another embodiment, the amino acid sequence from the E. coli CS3 protein is SEQ ID NO: 2.

An amino acid sequence from virtually any heterologous antigen may be expressed with an E. coli CS3 amino acid sequence to form a fusion protein. In one embodiment of the invention, the heterologous antigen is less than or equal to about 25 kDa, 50 kDa, 75 kDa, 100 kDa, 125 kDa, 150 kDa, 175 kDa, 200 kDa, 225 kDa, 250 kDa, 275 kDa or 300 kDa. A “heterologous antigen” refers to an immunogenic protein or peptide from a species different than the species of host cell in which the protein is expressed (e.g. if the host cell was an attenuated Salmonella, the heterologous antigen would be derived from a species other than Salmonella). In some embodiments, a heterologous antigen refers to an immunogenic protein or peptide from a different species than that from which the CS3 protein is naturally expressed (e.g. a species other than enterotoxigenic E. coli). A heterologous antigen may include, but is not limited to, bacterial antigens, viral antigens, parasitic antigens, eukaryotic antigens (e.g. cancer antigens), and therapeutic proteins. In one embodiment, the heterologous antigen is a fusion protein.

In one embodiment, the heterologous antigen (or antigens) is a bacterial antigen. Non-limiting examples of bacterial antigens include antigens from bacteria such as Bacillus spp. (e.g., Bacillus anthracis, B. cereus, B. subtilis and B. mycoides), Bordetella spp. (e.g., B. pertussis and B. recurrentis), Brucella spp. (e.g., B. abortus, B. canis, B. melitensis and B. suis), Campylobacter spp. (e.g., C. jejuni), Chlamydia spp. (e.g., C. pneumoniae, C. trachomatis and C. psittaci), Citrobacter spp. (e.g., C. freundii and C. diversus), Clostridium spp. (e.g., C. difficile, C. botulinum, C. perfringens and C. tetani), Corynebacterium spp. (e.g., C. diptheriae), Enterococcus spp. (e.g., E. faecalis and E. faecum), Escherichia spp. (e.g., enterotoxigenic E. coli, enteropathogenic E. coli, enterohemorrhagic E. coli and enteroinvasive E. coli), Enterobacter spp. (e.g., E. cloacae), Francisella spp. (e.g., F. tularensis), Helicobacter spp. (e.g., H. pylori), Klebsiella spp. (e.g., K. pneumoniae), Legionella spp. (e.g., L. pneumophilia), Leptospira spp. (e.g., L. interrogans), Listeria spp. (e.g., L. monocytogenes), Moraxella spp. (e.g., M. catarrhalis and M. lacunata), Mycobacterium spp. (e.g., M. leprae and M. tuberculosis), Mycoplasma spp. (e.g., M. pneumoniae), Neisseria spp. (e.g., N. gonorrhoeae and N. meningitidis), Plesiomonas spp. (e.g., P. shigelloides), Proteus spp. (e.g., P. mirabilis and P. vulgaris), Providencia spp. (e.g., P. alcalifaciens, P. stuartii and P. rettgeri), Pseudomonas spp. (e.g., P. aeruginosa), Rickettsia spp. (e.g., R. rickettsii and R. prowazekii), Salmonella spp. (e.g., S. typhimurium, S. typhi, S. choleraesuis and S. enteritidis), Serratia spp. (e.g., S. marcescens), Shigella spp. (e.g., S. dysenteriae, S. flexneri, S. boydii and S. sonnei), Spirillum spp. (e.g., S. minus), Staphylococcus spp. (e.g., S. aureus, S. epidermidis and S. saprophyticus), Streptococcus spp. (e.g., S. agalactiae, S. penumoniae and S. pyogenes), Treponema spp. (e.g., T. pallidum), Vibrio spp. (e.g., V. cholera, V. parahaemolyticus and V. vulnificus), Xanthomonas spp. (e.g., X. maltophilia) and Yersinia spp. (e.g., Y. pestis, Y. enterocolitica and Y. pseudotuberculosis).

In another embodiment, the bacterial heterologous antigen is a toxin or fragment thereof. For instance, the present invention includes heterologous antigens comprising C. difficile toxin A, C. difficile toxin A C-terminal repeat region, C. difficile toxin B, C. difficile toxin B C-terminal repeat region, C. botulinum toxin A, C. botulinum toxin B, C. botulinum toxin C, C. botulinum toxin D, C. botulinum toxin E, C. botulinum toxin F, C. botulinum toxin LH_{N} fragment (e.g., LH_{N}/A, LH_{N}/B, LH_{N}/C, LH_{N}/D, LH_{N}/E and LH_{N}/F), Bacillus anthracis toxin and components (e.g., anthrax toxin, anthrax lethal factor, anthrax protective antigen, anthrax edema factor, EF toxin), C. diphtheriae toxin, C. diphtheriae toxin fragment A, C. diphtheriae toxin fragment B, V. cholerae toxin (e.g., Ctx toxin), E. coli heat stable toxin, E. coli heat stable toxin subunit B, E. coli heat labile toxin, E. coli 0157:H7 toxins (i.e., verotoxin 1 and verotoxin 2), Shiga toxin (e.g., S. dysenteriae toxin), B. pertussis Ptx toxin, B. pertussis AC toxin, C. tetani toxin, S. aureus exofoliatin B, S. aureus alpha toxin, S. aureus leukocidin, C. perfringens perfringiolysin O toxin, C. perfringens enterotoxin, E. coli hemolysin, L. monocytogenes listeriolysin, S. pyogenes streptolysin O, streptococcal pyrogenic toxin (SPE), staphylococcal enterotoxin, S. aureus toxic shock syndrome toxin (TSST-1), and Pseudomonas exotoxin A and immunogenic fragments thereof. In some embodiments, the bacterial toxin may be mutated to reduce or prevent toxin activity.

In another embodiment of the invention, the heterologous antigen (or antigens) is a viral antigen. For instance, the invention includes viral antigens from Bunyavirus (e.g., Bunyamera virus, Bwamba virus, Hantavirus, Dugbe virus, Orepuche virus, Nairovirus, Naples and Sicilian Sandfly Fever virus, Rift Valley Fever virus), Coronaviruses (e.g., Human Coronaviruses 229E, NL63 and OC43, Severe Acute Respiratory Syndrome Coronavirus and Infectious Bronchitis virus), Erythroviruses (e.g., Parvovirus B19), Filoviruses (e.g., Marburg virus, Ebola virus), Flaviviruses (e.g., West Nile virus, Yellow Fever virus, Dengue virus, Hepatitis C virus), Herpesviruses (e.g., Herpes Simplex-1 virus, Herpes Simplex-2 virus, Varicella Zoster virus, Roseolovirus, Cytomegalovirus and Epstein-Barr virus), Lentiviruses (e.g., HIV and other Retroviruses), Orthohepadnavirus (e.g., Hepatitis B virus), Orthomyxoviruses (e.g., Influenza A, Influenza B, Influenza C virus, Thogoto virus, Dhori virus), Papillomaviruses (e.g., Human Papilloma virus types 6, 11, 16, 18, 31, 33, 35, 39, 42, 43, 44, 45, 51, 52, 56, 58, 59 and 68), Paramyxoviruses (e.g., Measles virus, Human Parainfluenza virus 1 and 3, Respiratory Syncytial virus, Hendravirus, Nipah virus, Mumps virus, Menangle virus, Tioman virus), Parvoviruses, Picornavirues (e.g., Poliovirus, Hepatitis A virus, Human Enteroviruses and Human Rhinoviruses), Poxviruses (e.g., Smallpox virus, Vaccinia virus and Molluscum contagiosum virus), Rhabdoviruses (e.g., Rabies virus), Rhinovirus, Rotavirus and Togavirus (e.g., Rubella virus).

In another embodiment of the invention, the heterologous antigen (or antigens) is a fungal antigen. Examples of fungal antigens include, but are not limited to, antigens from Absidia spp. (e.g. Absidia corymbifera), Ajellomyces spp. (e.g. Ajellomyces capsulatus, Ajellomyces dermatitidis), Arthroderma spp. (e.g. Arthroderma benhamiae, Arthroderma fulvum, Arthroderma gypseum, Arthroderma incurvatum, Arthroderma otae, Arthroderma vanbreuseghemii), Aspergillus spp. (e.g. Aspergillus fumigatus, Aspergillus niger), Candida spp. (e.g. Candida albicans, Candida albicans var. stellatoidea, Candida dublinensis, Candida glabrata, Candida guilliermondii, Candida krusei (Issatschenkia orientalis), Candida parapsilosis, Candida pelliculosa, Candida tropicalis), Coccidioides (e.g. Coccidioides immitis), Cryptococcus spp. (e.g. Cryptococcus neoformans), Histoplasma spp. (e.g. Histoplasma capsulatum (Ajellomyces capsulatus), Microsporum spp. (e.g. Microsporum canis (Arthroderma otae), Microsporum fulvum (Arthroderma fulvum), Microsporum gypseum), Genus Pichia (e.g. Pichia anomala, Pichia guilliermondii), Pneumocystis (e.g. Pneumocystis jirovecii), Cryptosporidium, Malassezia furfur, Paracoccidiodes spp. (e.g. Paracoccidioides brasiliensis), Blastomyces dermatitidis, Mucorales spp., and Sporothrix schenckii.

In yet another embodiment, the heterologous antigen (or antigens) is a parasitic antigen. For instance, the invention includes parasitic antigens from Plasmodium spp. (e.g., P. falciparum, P. vivax, P. ovale and P. malariae), Trypanosome spp., Trichomonas spp., Toxoplasma spp., Giardia spp., Boophilus spp., Babesia spp., Entamoeba spp., Eimeria spp., Leishmania spp., Schistosome spp., Brugia spp., Fascida spp., Dirofilaria spp., Wuchereia spp., and Onchocerea spp.

In some embodiments, the heterologous antigen (or antigens) may be a eukaryotic antigen or a therapeutic peptide. For example, the heterologous antigen or antigens may include cancer antigens (e.g., Alpha-fetoprotein, NY-ESO-1, MAGE-A, Her-2/neu, p53, MelanA/MART-1, cancer antigen 125, cancer antigen 15-3, carcinoembryonic antigen, GAGE, SSX, LAGE-1 and cancer antigen 19-9, cancer antigen 72-4, cancer antigen 195, adenocarcinoma antigen ART1, squamous cell carcimona antigen 1, squamous cell carcimona antigen 2, DUPAN-2, neuro-oncological ventral antigen 2, CTCL tumor antigen sel-1, CTCL tumor antigen sel4-3, CTCL tumor antigen se20-4, CTCL tumor antigen se33-1, CTCL tumor antigen se37-2, CTCL tumor antigen se57-1, CTCL tumor antigen se-89-1, prostate-specific membrane antigen, hepatocellular carcinoma antigen gene 520, or tumor-associated antigen CO-029), autoimmune antigens, or other eukaryotic pathological proteins. The CS3 expression system may also be used to deliver therapeutic peptides to a subject such as a cytokine (e.g. IL-2, IL-4, IL-5, IL-6, IL-10, IL-12, IFN-gamma or granulocyte macrophage colony stimulating factor).

In other embodiments of the invention, the fusion protein further comprises a linker amino acid sequence. The linker amino acid sequence may be positioned between the CS3 export amino acid sequence and the heterologous antigen amino acid sequence. In another embodiment, the linker amino acid sequence is located between two heterologous antigens. The linker amino acid sequence may be at least about 2, 3, 4, 5, 6, 7, 8, 9, 10, 15, or about 20 amino acids in length. A linker amino acid sequence may be incorporated into the fusion protein to facilitate proper folding of the heterologous antigen. In one embodiment, the linker amino acid sequence may contain one or more amino acids flanking the export signal sequence in the CS3 protein (SEQ ID NOS: 17 and 19). In another embodiment, the nucleic acid encoding the linker amino acid sequence contains at least one site for a restriction enzyme. In another embodiment, the linker amino acid sequence is comprised of proline, glycine, alanine, serine, threonine, and/or asparagine residues. Some exemplary amino acid linker sequences include, but are not limited to, (as represented by the single letter amino acid code) A-A-P-G (SEQ ID NO: 20); A-A-G-P (SEQ ID NO: 21); P-G; and G-P. Linker amino acid sequences encoding proline and glycine residues may be preferred in some embodiments.

The present invention also encompasses variants of the CS3 protein export signal and/or the heterologous antigens incorporated into the fusion proteins. The variants may contain one or more alterations in the amino acid sequences of the constituent proteins. The term “variant” with respect to a polypeptide refers to an amino acid sequence that is altered by one or more amino acids with respect to a wild-type sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

Variants of the CS3 protein or fragments thereof should retain the ability to export fusion proteins containing the CS3 variant sequence to the outer surface of the host cell. In one embodiment, the CS3 variant contains a sequence that is at least about 75, 80, 85, 90, 91, 92, 93, 94, 95, 96, 97, 98, or 99% identical to the polypeptide of SEQ ID NO: 8. In another embodiment, the CS3 variant contains a sequence that is at least about 75, 80, 85, 90, 91, 92, 93, 94, 95, 96, 97, 98, or 99% identical to the polypeptide of SEQ ID NO: 17 or 19. In another embodiment, the CS3 variant contains a sequence that is at least about 75, 80, 85, 90, 91, 92, 93, 94, 95, 96, 97, 98, or 99% identical to the polypeptide of SEQ ID NO: 2. Variant sequences may be shorter or longer in length as compared to the wild-type sequence. The percentage sequence identity is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of sequence identity. Percentage of sequence identity is determined by comparing two optimally aligned sequences over a comparison window. In order to optimally align sequences for comparison, the portion of a polynucleotide or polypeptide sequence in the comparison window may comprise additions or deletions termed gaps while the reference sequence is kept constant. An optimal alignment is that alignment which, even with gaps, produces the greatest possible number of “identical” positions between the reference and comparator sequences. Percentage sequence identity between two sequences may be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option.

The genetic constructs of the invention comprise a promoter operably linked to a nucleic acid encoding a fusion protein described herein. The phrase “operably linked” means that the promoter is in the correct location and orientation in relation to a nucleic acid to control the initiation of transcription by RNA polymerase and expression of the nucleic acid. In some embodiments, the promoter is an inducible promoter that is operative under specific physiological conditions.

In certain embodiments, the inducible promoter is a prokaryotic inducible promoter. For example, the inducible promoter of the invention includes a Gram negative bacterium promoter, including, but not limited to, a Salmonella promoter.

In some embodiments, the inducible promoter is an in vivo inducible promoter. For instance, in some embodiments, the inducible promoter directs expression of a fusion protein described herein in the gastrointestinal tract of the host. In still other embodiments, the inducible promoter directs expression of the fusion protein within the gastrointestinal tract and immune cells (e.g. macrophages) of the host.

The inducible promoter may direct expression of a fusion protein (e.g. E. coli CS3 export signal sequence fused to at least one heterologous antigen amino acid sequence) under acidic conditions. For instance, in some embodiments, the inducible promoter directs expression of a fusion protein at a pH of less than or about pH 7, including at a pH of less than or about pH 6, pH 5, pH 4, pH 3 or pH 2.

The promoter of the invention can also be induced under conditions of low phosphate concentrations. In one embodiment, the promoter is induced in the presence of low pH and low phosphate concentration such as the conditions that exist within macrophages. In certain embodiments, the promoter of the invention is induced under highly oxidative conditions such as those associated with macrophages.

In some embodiments, the promoter directs the expression of the fusion protein under conditions and/or locations in a host so as to induce systemic and/or mucosal immunity against the antigen. Such promoters may include, but are not limited to, the ssaG, ssrA, sseA, pagC, nirB and katG promoters of Salmonella. The in vivo inducible promoter may be as described in WO 02/072845, which is hereby incorporated by reference in its entirety.

In preferred embodiments, the promoter is a Salmonella ssaG promoter. The ssaG promoter is normally located upstream of the start codon for the ssaG gene, and may comprise the nucleotide sequence of SEQ ID NO: 15 listed below.

(SEQ ID NO: 15)
CTCGAGATTG CCATCGCGGA TGTCGCCTGT CTTATCTACC ATCATAAACA TCATTTGCCT
ATGGCTCACG ACAGTATAGG CAATGCCGTT TTTTATATTG CTAATTGTTT CGCCAATCAA
CGCAAAAGTA TGGCGATTGC TAAAGCCGTC TCCCTGGGCG GTAGATTAGC CTTAACCGCG
ACGGTAATGA CTCATTCATA CTGGAGTGGT AGTTTGGGAC TACAGCCTCA TTTATTAGAG
CGTCTTAATG ATATTACCTA TGGACTAATG AGTTTTACTC GCTTCGGTAT GGATGGGATG
GCAATGACCG GTATGCAGGT CAGCAGCCCA TTATATCGTT TGCTGGCTCA GGTAACGCCA
GAACAACGTG CGCCGGAGTA ATCGTTTTCA GGTATATACC GGATGTTCAT TGCTTTCTAA
ATTTTGCTAT GTTGCCAGTA TCCTTACGAT GTATTTATTT TAAGGAAAAG CCAT

In this context, the term “ssaG promoter” includes promoters having similar or modified sequences, and similar or substantially identical promoter activity, as the wild-type ssaG promoter, and particularly with respect to its ability to induce expression in vivo. Similar or modified sequences may include nucleotide sequences with high identity to SEQ ID NO: 15, such as nucleotide sequences having at least about 50%, 60%, 70%, 80%, 90%, or 95% identity to SEQ ID NO: 15, as well as functional fragments, including functional fragments with high identity to corresponding functional fragments of SEQ ID NO: 15. In certain embodiments, the functional ssaG promoter fragment comprises at least about 30 nucleotides, at least about 40 nucleotides, or at least about 60 nucleotides. In one embodiment of the invention, the ssaG promoter is optimized for function in a host cell.

General texts which describe molecular biological techniques, which are applicable to the present invention, such as cloning, mutation, construction of vectors and the like, include Berger and Kimmel, Guide to Molecular Cloning Techniques, Methods in Enzymology volume 152 Academic Press, Inc., San Diego, Calif (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutating of the ETEC CS3 proteins and sequences for heterologous antigens, and are all herein incorporated by reference. Thus, the invention also encompasses using known methods of protein engineering and recombinant DNA technology to improve or alter the characteristics of the fusion proteins expressed from the genetic constructs of the invention. Various types of mutagenesis can be used to produce and/or isolate variant nucleic acids that encode for protein molecules and/or to further modify/mutate the proteins in the fusion proteins of the invention. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like.

E. coli CS3 polypeptides and antigens from heterologous organisms can be cloned using methods known in the art. For example, the gene encoding the ETEC CS3 protein can be isolated by RT-PCR from polyadenylated mRNA extracted from an enterotoxigenic strain of bacteria. The resulting product gene can be cloned as a DNA insert into a vector.

Alternatively, if the nucleotide sequence or amino acid sequence of the heterologous antigen of interest is known, the polynucleotide or peptide can be chemically synthesized. The term “vector” refers to the means by which a nucleic acid can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating.

The present invention also includes recombinant fusion proteins and polynucleotides encoding the same. The recombinant fusion proteins of the invention comprise an amino acid sequence from E. coli CS3 protein and an amino acid sequence from at least one heterologous antigen. The CS3 protein may be from an enterotoxigenic species of E. coli, i.e. “ETEC”. The amino acid sequence from the E. coli CS3 protein incorporated into the fusion protein may be the full length amino acid sequence of the protein or an amino acid sequence of a fragment consisting essentially of an export signal. The recombinant fusion proteins of the invention are useful for inducing an immune response and/or generating antibodies against the fusion protein in a subject, such as a human subject.

The fusion protein may be cleaved in the host cell in which it is expressed prior to being secreted or displayed on the outer surface of the host cell. Signal sequences are often cleaved from a protein after targeting the protein to the correct cellular location. In some embodiments, the CS3 export signal sequence is completely removed from the fusion protein prior to the export of the fusion protein from the cell. In other embodiments, the CS3 export signal is partially cleaved from the fusion protein prior to the export of the fusion protein from the cell. That is, the fusion protein may retain 1, 2, 3, 4, 5, 6, 7, 8, 9, or 10 amino acids of the CS3 export signal sequence after export from the cell. Thus, the present invention also includes a recombinant fusion protein comprising a heterologous antigen amino acid sequence fused to a partial CS3 export signal sequence (e.g. 1, 2, 3, 4, 5, 6, 7, 8, 9, or 10 amino acids of a CS3 export signal sequence).

In some embodiments, the recombinant fusion proteins of the invention may comprise an antigen from enterotoxigenic E. coli (ETEC) fused to a CS3 peptide containing an export signal. For example, the recombinant fusion protein may comprise amino acid sequences from ETEC heat labile toxin and heat stable toxin fused to a CS3 peptide. In one embodiment, the recombinant fusion protein has an amino acid sequence of SEQ ID NO: 12. In another embodiment, the recombinant fusion protein has an amino acid sequence of SEQ ID NO: 14.

The invention includes an isolated recombinant fusion protein. The recombinant fusion protein can be isolated by methods known in the art. An isolated recombinant fusion protein can be purified, for instance, substantially purified. An isolated recombinant fusion protein can be purified by methods generally known in the art, for instance, by electrophoresis (e.g., SDS-PAGE), filtration, chromatography, centrifugation, and the like. A substantially purified recombinant fusion protein can be at least about 60% purified, 65% purified, 70% purified, 75% purified, 80% purified, 85% purified, 90% purified or 95%, 96%, 97%, 98%, 99%, or greater purified (i.e. 100% purified).

The invention further provides a polynucleotide encoding the recombinant fusion proteins of the invention. Such recombinant fusion proteins may be under the control of an inducible promoter as described, such as a Salmonella ssaG promoter, for example. The polynucleotide may be designed for integration at, or integrated at, an aroC and/or ssaV gene deletion site of a Salmonella host cell. In some embodiments, the polynucleotide of the invention is a suicide vector for constructing an attenuated microorganism of the invention. The invention includes an isolated and/or purified polynucleotide. By “isolated,” it is meant that the polynucleotide is substantially free of other nucleic acids, e.g., at least about 20% pure, preferably at least about 40% pure, more preferably about 60% pure, even more preferably about 80% pure, most preferably about 90% pure, and even most preferably about 95% pure, as determined by agarose gel electrophoresis. A polynucleotide can be isolated or purified by methods generally known in the art.

The present invention also encompasses a method for exporting one or more heterologous antigens from a cell. In one embodiment, the method comprises expressing in a cell a genetic construct encoding at least one fusion protein, wherein said fusion protein comprises an amino acid sequence from E. coli CS3 protein consisting essentially of an export signal fused to at least one heterologous antigen amino acid sequence. In another embodiment, the fusion protein is expressed from an inducible promoter as described above. In some embodiments, the expression of the fusion protein may be induced in vivo. In a particular embodiment, the fusion protein is expressed from a Salmonella ssaG promoter. Any heterologous antigen, such as those described herein, may be used according to the inventive method.

Several different cell types are suitable for use in the methods of the invention. Exemplary cell types include yeast (e.g. S. cerevisiae, Kluyveromyces lactis, species of Candida including C. albicans and C. glabrata, Aspergillus nidulans, Schizosaccharomyces pombe, Pichia pastoris, and Yarrowia lipolytica), insect (e.g. Spodoptera frugiperda cells (Sf9, Sf21), Trichoplusia ni cells (High Five cells), and Drosophila S2 cells), amphibian (e.g. Xenopus laevis oocytes), avian, plant, and C. elegans (or nematode), and bacterial cells, such as E. coli, B. subtilis, mycobacteria, and Salmonella. In a preferred embodiment, the host cell is a bacterial cell. In another preferred embodiment, the host cell is an attenuated Salmonella. The attenuated Salmonella may comprise one or more of the attenuating mutations described above, such as a deletion or inactivation of a SPI-2 gene (e.g. ssaV) and/or a deletion or inactivation of a gene involved in the biosynthesis of aromatic compounds (e.g. aroC).

The nucleotide sequences encoding the CS3 secretion tag and/or fusion protein may be codon optimized to facilitate expression of the fusion protein in particular host cells. By way of example, sequences of heterologous antigens derived from prokaryotic pathogens may be codon optimized for expression in heterologous prokaryotic host cells. In one embodiment, nucleotide sequences are codon optimized for expression in Salmonella spp. The invention also includes, for instance, nucleic acids encoding fusion proteins that are codon optimized for expression in S. enterica serovars such as S. enterica Typhimurium, S. enterica Typhi, and S. enterica Enteritidis. The invention also includes nucleic acids that are codon optimized for expression in Vibrio spp. host cells, Listeria spp.host cells, Shigella spp. host cells or E. coli host cells. Similarly, promoter selection may also be optimized for expression in certain host cells. Codon optimization and promoter selection for expression in particular types of host cells are within the skill of the ordinary artisan.

Methods of delivering a genetic construct to a host cell are also within the skill and knowledge of one of ordinary skill in the art. Such methods include, but are not limited to, calium or rubidium chloride mediate transformation, calcium phosphate co-precipitation, electroporation, conjugation, transduction, microinjection, lipofection, and transfection employing polyamine transfection reagents. In one embodiment, the fusion protein is secreted from the cell following delivery of the genetic construct.

Export of the fusion proteins from a host cell may be determined by a number of methods. Some methods include immunolabeling of intact host cells, western blot analysis of cell culture supernatant, or fractionation of host cellular lysates followed by western blot analysis to detect the location of the expressed fusion protein. Other methods known to those skilled in the art are also suitable for analysis of the fusion protein trafficking.

The present invention also provides an attenuated microorganism comprising a genetic construct as described herein. As used herein, the term “attenuated” refers to a microorganism, such as a bacterium, that has been genetically modified so as to not cause illness in a human or animal model. In one embodiment, an “attenuated” microorganism also refers to a microorganism that has been genetically modified so that is unable to replicate and/or grow in a host organism.

In one embodiment of the invention, the attenuated microorganism comprises a genetic construct comprising a promoter, nucleic acid sequence encoding a CS3 polypeptide and nucleic acid sequence encoding one or more heterologous antigens. For instance, the invention comprises a genetic construct comprising a cs3 nucleic acid sequence such as that of SEQ ID NOs.: 1, 7 or 9, or a nucleic acid sequence with at least about 85%, 90% or 95% identity to the nucleic acid sequence of SEQ ID NOs.: 1, 7 or 9. In one embodiment of the invention, the attenuated microorganism, i.e., host cell, also comprises a nucleic acid encoding one or more linkers.

In one embodiment, the attenuated microorganism comprises a gene express cassette comprising the promoter, nucleic acid encoding a CS3 polypeptide, nucleic acid encoding one or more heterologous antigens, and, optionally, one or more linkers. The gene expression cassette may be carried on a plasmid or may be integrated into the chromosome of the host cell.

In one embodiment, the attenuated microorganism comprises a genetic construct encoding a fusion protein under the control of a promoter, wherein the fusion protein comprises an amino acid sequence from E. coli CS3 polypeptide consisting essentially of an export signal fused to at least one heterologous antigen amino acid sequence. In another embodiment, the microorganism expresses the fusion protein on its surface. In yet another embodiment, the microorganism secretes the fusion protein.

In some embodiments of the invention, the heterologous antigens are presented to the host immune system as part of a fusion protein, as described above, by means of a live, attenuated microorganism or bacterial vaccine platform, such as an attenuated Gram negative bacterial vaccine platform. As used herein, the term “bacterial vaccine platform” refers to an attenuated microorganism that is used to express a heterologous antigen in a host for the purpose of eliciting a protective immune response to the heterologous antigen. The attenuated microorganisms, including attenuated Salmonella enterica serovars, provided herein are suitable bacterial vaccine platforms. In one embodiment, the bacterial vaccine platform is the Salmonella typhi spi-VEC™ live attenuated bacterial vaccine platform (Emergent Product Development UK, UK). Other exemplary microbial platforms include Vibrio cholerae, Shigella spp., Listeria monocytogenes, Mycobacterium bovis, Yersinia pseudotuberculosis, Bordetella pertussis, and Salmonella spp., as well as others described in U.S. Pat. No. 5,877,159, which is hereby incorporated by reference in its entirety. In certain embodiments, the bacterial vaccine platform or attenuated microorganism is an attenuated Salmonella enterica serovar, for instance, S. enterica serovar Typhi, S. enterica serovar Typhimurium, S. enterica serovar Paratyphi, S. enterica serovar Enteritidis, S. enterica serovar Choleraesuis, S. enterica serovar Gallinarum, S. enterica serovar Dublin, S. enterica serovar Hadar, S. enterica serovar Infantis and S. enterica serovar Pullorum.

Generally, the microorganism carries one or more gene deletions or mutations, which attenuates the microorganism. In some embodiments, the microorganism is attenuated by deletion of all or a portion of a gene(s) associated with pathogenicity. In certain embodiments, a gene expression cassette expressing a fusion protein comprising an E. coli CS3 export sequence and a heterologous antigen may be inserted into the site of the deletion of one or more genes associated with pathogenicity. Alternatively, the pathogenicity gene(s) may be inactivated, for example, by mutation in an upstream regulatory region or upstream gene so as to disrupt expression of the pathogenesis-associated gene, thereby leading to attenuation. For example, a gene may be inactivated by an insertional mutation. In one embodiment, the insertional mutation is caused by the insertion of a gene expression cassette comprising the promoter and nucleic acid encoding the fusion protein of the invention.

In other embodiments, the attenuated microorganism may be an attenuated Gram negative bacterium as described in U.S. Pat. Nos. 6,342,215; 6,756,042 and 6,936,425, each of which is hereby incorporated by reference in its entirety. For example, the microorganism may be an attenuated Salmonella spp. (e.g., S. enterica Typhi or S. enterica Typhimurium) comprising a first deletion or inactivation in a gene located within the Salmonella pathogenicity island 2 (SPI-2). The present invention includes an attenuated Salmonella spp. with more than one deleted or inactivated SPI-2 genes.

SPI-2 is one of more than two pathogenicity islands located on the Salmonella chromosome. SPI-2 comprises several genes that encode a type III secretion system involved in transporting virulence-associated proteins, including SPI-2 so-called effector proteins, outside of the Salmonella bacteria and potentially directly into target host cells such as macrophages. SPI-2 apparatus genes encode the secretion apparatus of the type III system. SPI-2 is essential for the pathogenesis and virulence of Salmonella in the mouse. S. typhimurium SPI-2 mutants are highly attenuated in mice challenged by the oral, intravenous and intraperitoneal routes of administration.

Infection of macrophages by Salmonella activates the SPI-2 virulence locus, which allows Salmonella to establish a replicative vacuole inside macrophages, referred to as the Salmonella-containing vacuole (SCV). SPI-2-dependent activities are responsible for SCV maturation along the endosomal pathway to prevent bacterial degradation in phagolysosomes, for interfering with trafficking of NADPH oxidase-containing vesicles to the SCV, and remodeling of host cell microfilaments and microtubule networks. See, for instance, Vazquez-Torres et al., Science, Vol. 287:1655-1658 (2000), Meresse et al., Cell Microbiol., Vol. 3:567-577 (2001) and Guignot et al., J. Cell Sci., Vol. 117:1033-1045 (2004), each of which is herein incorporated by reference in its entirety. Salmonella SPI-2 mutants are attenuated in cultured macrophages (see, for instance, Deiwick et al., J. Bacteriol., Vol. 180(18):4775-4780 (1998) and Klein and Jones, Infect. Immun., Vol. 69(2):737-743 (2001), each of which is herein incorporated by reference in its entirety). Specifically, Salmonella enterica SPI-2 mutants generally have a reduced ability to invade macrophages as well as survive and replicate within macrophages.

The deleted or inactivated SPI-2 gene may be, for instance, an apparatus gene (ssa), an effector gene (sse), a chaperone gene (ssc) or a regulatory gene (ssr). In some embodiments, the attenuated Salmonella microorganism is attenuated via a deletion or inactivation of a SPI-2 apparatus gene, such as those described in Hensel et al., Molecular Microbiology, Vol. 24(1):155-167 (1997) and U.S. Pat. No. 6,936,425, each of which is herein incorporated by reference in its entirety. In specific embodiments, the attenuated Salmonella carries a deletion or inactivation of at least one gene associated with pathogenesis selected from ssaV, ssaJ, ssaU, ssaK, ssaL, ssaM, ssaO, ssaP, ssaQ, ssaR, ssaS, ssaT, ssaU, ssaD, ssaE, ssaG, ssaI, ssaC (spiA) and ssaH. In a preferred embodiment, the attenuated Salmonella may carry a deletion and/or inactivation of the ssaV gene. Alternatively, or in addition, the microorganism may carry a mutation within an intergenic region of ssaJ and ssaK. The attenuated Salmonella may of course carry additional deletions or inactivations of the foregoing genes, such as two, three, or four genes.

In other embodiments, the attenuated Salmonella microorganism comprises a deletion or inactivation of a SPI-2 effector gene. For example, the attenuated Salmonella may comprise a deletion or inactivation of at least one gene selected from sseA, sseB, sseC, sseD, sseE, sseF, sseG, sseL and spiC (ssaB). The sseB gene is necessary to prevent NADPH oxidase localization and oxyradical formation at the phagosomal membrane of macrophages. SseD is involved in NADPH oxidase assembly. SpiC is an effector protein that is translocated into Salmonella-infected macrophages and interferes with normal membrane trafficking, including phagosome-lysosome fusion. See, for example, Hensel et al., Mol. Microbiol., Vol. 30:163-174 (1998); Uchiya et al., EMBO J., Vol. 18:3924-3933 (1999); and Klein and Jones, Infect. Immun., Vol. 69(2):737-743 (2001), each of which is herein incorporated by reference in its entirety. The attenuated Salmonella may of course carry additional deletions or inactivations of the foregoing genes, such as two, three, or four genes.

In still other embodiments, the attenuated Salmonella microorganism comprises a deleted or inactivated ssr gene. For instance, the attenuated Salmonella may comprise a deletion or inactivation of at least one gene selected from ssrA (spiR) and ssrB. ssrA encodes a membrane-bound sensor kinase (SsrA), and ssrB encodes a cognate response regulator (SsrB). SsrB is responsible for activating transcription of the SPI-2 type III secretion system and effector substrates located outside of SPI-2. See, for instance, Coombes et al., Infect. Immun., Vol 75(2):574-580 (2007), which is herein incorporated by reference in its entirety.

In some embodiments, the attenuated Salmonella comprises an inactivated SPI-2 gene encoding a chaperone (ssc). For instance, the attenuated Salmonella may comprise a deletion or inactivation of sscA and/or sscB. See, e.g., U.S. Pat. No. 6,936,425, which is herein incorporated by reference in its entirety.

The attenuated Salmonella may comprise one or more additional independently attenuating mutations outside of the SPI-2 region. For example, the attenuated Salmonella may carry an “auxotrophic mutation,” i.e. a mutation that is essential to a biosynthetic pathway. The biosynthetic pathway is generally one present in the microorganism, but not present in mammals, such that the mutants cannot depend on metabolites present in the treated patient to circumvent the effect of the mutation. In some embodiments, the present invention includes an attenuated Salmonella with a deleted or inactivated gene necessary for the biosynthesis of aromatic amino acids. Exemplary genes for the auxotrophic mutation in Salmonella, include an aro gene e.g., aroA, aroC, aroD, and aroE. In one embodiment, the attenuated Salmonella has a deletion or inactivation of the aroC gene. In another embodiment, the invention comprises a Salmonella SPI-2 mutant (e.g. mutation in ssaV gene) comprising an attenuating mutation in the aroC gene. In addition to aro gene mutations, the present invention includes an attenuated Salmonella with the deletion or inactivation of a purA, purE, asd, guaB, guaA, cya, clpP , clpX and/or crp gene.

In another embodiment, the attenuated Salmonella SPI-2 mutant also comprises at least one additional deletion or inactivation of a gene in the Salmonella Pathogenicity Island I region (SPI-1). In still another embodiment, the Salmonella SPI-2 mutant comprises at least one additional deletion or inactivation of a gene outside of the SPI-2 region which reduces the ability of Salmonella to invade a host cell and/or survive within macrophages. For instance, the second mutation may be the deletion or inactivation of a rec or sod gene. In yet another embodiment, the Salmonella spp. comprises the deletion or inactivation of transcriptional regulator that regulates the expression of one or more virulence genes (including, for instance, genes necessary for surviving and replicating within macrophages). For instance, the Salmonella SPI-2 mutant may further comprise the deletion or inactivation of one or more genes selected from the group consisting of phoP, phoQ, rpoS and slyA.

In some embodiments, the attenuated microorganism is a Salmonella microorganism having attenuating mutations in a SPI-2 gene (e.g., ssa, sse, ssr or ssc gene) and an auxotrophic gene located outside of the SPI-2 region. In one embodiment, the attenuated microorganism is a Salmonella enterica serovar comprising a deletion or inactivation of an ssa, sse and/or ssr gene and an auxotrophic gene. For instance, the invention includes an attenuated Salmonella enterica serovar with deletion or inactivating mutations in the ssaV and aroC genes (for example, a microorganism derived from Salmonella enterica Typhi ZH9, as described in U.S. Pat. No. 6,756,042, which is hereby incorporated by reference in its entirety) or ssaJ and aroC genes.

The present invention also provides diagnostic methods using the attenuated microorganisms described herein. In one embodiment, the method comprises contacting an attenuated microorganism of the invention with a putative binding partner, wherein the attenuated microorganism secretes or displays a biosensor protein; and measuring a signal from the biosensor protein (e.g. a fluorescent signal), wherein a change in the biosensor signal is indicative of the binding of the putative binding partner to the secreted or displayed biosensor protein. Biosensor proteins are known in the art, and typically produce a change in fluorescence upon a conformational shift of the protein induced by the binding of a substance. See, e.g. U.S. Pat. No. 5,998,204; U.S. Pat. No. 5,981,200; and WO 06/044611, all of which are herein incorporated by reference in their entireties. Use of attenuated microorganisms exporting such biosensor proteins to detect particular antigens or substances in vivo is also contemplated by the invention.

The present invention also includes pharmaceutical compositions comprising the attenuated microorganisms disclosed herein. Pharmaceutical compositions include, but are not limited to vaccine and therapeutic compositions. Pharmaceutical compositions include compositions that can be administered pre-exposure or post-exposure to a pathogen to prevent or ameliorate an infection or disease associated with an infection. Pharmaceutical compositions of the invention include compositions that can be administered to prevent or treat an infection or disease (including, but not limited to a cancer). The terms pharmaceutical composition, vaccine and therapeutic composition are used interchangeably herein.

In one embodiment, the pharmaceutical composition comprises an attenuated microorganism and a pharmaceutically acceptable carrier, wherein said microorganism comprises a genetic construct comprising a promoter operably linked to a nucleic acid encoding a fusion protein, said fusion protein comprising an amino acid sequence from E. coli CS3 protein and at least one heterologous antigen amino acid sequence. In some embodiments, the amino acid sequence from E. coli CS3 protein consists essentially of an export signal. In other embodiments, the amino acid sequence from E. coli CS3 is the amino acid sequence of the full length protein.

In some embodiments, the composition comprises an attenuated Salmonella that expresses the fusion protein on its surface. In certain embodiments, the fusion protein is secreted from the attenuated Salmonella. The composition can be formulated for oral, parenteral, topical, intratumoral, intramuscular, intravenous, subcutaneous, intraperitoneal, transdermal or buccal, administration to a subject, such as a human subject. Preferably, the attenuated microorganism contained within the pharmaceutical composition induces a mucosal immune response to the fusion protein when administered orally to the subject.

As detailed above, the attenuated microorganism may express a fusion protein comprising an amino acid sequence of any heterologous antigen fused to an export signal sequence from an E. coli CS3 protein. The invention includes compositions comprising such attenuated microorganisms for protection against the pathogens from which the heterologous antigens are derived. A “pathogen” is an infectious agent, such as a bacterium or virus, that causes disease or illness in a host. Non-limiting examples of pathogens which the pharmaceutical compositions of the invention may be effective in preventing or treating include, but are not limited to, Mycobacterium tuberculosis, Streptococcus, Pseudomonas, Shigella, Campylobacter and Salmonella, enterotoxigenic E. coli (ETEC), Clostridium difficile, Chlamydia trachomatis, Vibrio cholera, Clostridium botulinum, Bacillus anthracis, Helicobacter pylori, Yersinia pestis, Adenoviruses, Picornaviruses, Herpes viruses, Hepadnaviruses, Flaviviruses, Retroviruses (e.g. human immunodeficiency virus), Orthomyxoviruses, Paramyxoviruses, Papovaviruses, Rhabdoviruses, Togaviruses, Plasmodium falciparum, Plasmodium vivax, Plasmodium guale, and Plasmodium malariae. In one embodiment, the composition comprises an attenuated microorganism, such as an attenuated Salmonella, expressing a fusion protein comprising an export signal sequence from E. coli CS3 protein fused to amino acid sequences from heat stable and heat labile enterotoxins from enterotoxigenic E. coli (ETEC). In some embodiments, the ETEC heat stable and subunits of heat labile enterotoxins may be fused to create an ETEC fusion antigen. The attenuated microorganisms of the compositions may express more than one fusion protein, wherein each fusion protein contains amino acid sequences from different heterologous antigens. An amino acid sequence from any heterologous antigen from a pathogen is contemplated for use in the compositions of the invention.

The composition may comprise the attenuated microorganism as described, and a pharmaceutically acceptable carrier, for instance, a pharmaceutically acceptable vehicle, excipient and/or diluent. The term “pharmaceutically acceptable” means approved by a regulatory agency of the Federal or a state government or listed in the U.S. Pharmacopeia or other generally recognized pharmacopeia for use in animals, and more particularly in humans. The pharmaceutically acceptable carrier can be any solvent, solid or encapsulating material in which the pharmaceutical composition can be suspended or dissolved. The pharmaceutically acceptable carrier is non-toxic to the inoculated individual and compatible with the live, attenuated microorganism.

Suitable pharmaceutical carriers are known in the art, and include, but are not limited to, liquid carriers such as saline and other non-toxic salts at or near physiological concentrations. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Examples of suitable pharmaceutical vehicles, excipients, and diluents are described in “Remington's Pharmaceutical Sciences” by E. W. Martin, which is hereby incorporated by reference in its entirety.

In one embodiment of the invention, the composition comprises one or more of the following carriers: disodium hydrogen phosphate, soya peptone, potassium dihydrogen phosphate, ammonium chloride, sodium chloride, magnesium sulphate, calcium chloride, sucrose, sterile saline and sterile water. In another embodiment of the invention, the composition comprises an attenuated Salmonella enterica serovar (e.g., Typhi or Typhimurium) with deleted or inactivated SPI-2 (e.g., ssaV) and aroC genes and one or more genetic constructs comprising a nucleic acid encoding a fusion protein as described herein under the control of an in vivo inducible promoter (e.g., ssaG promoter) and a carrier comprising disodium hydrogen phosphate, soya peptone, potassium dihydrogen phosphate, ammonium chloride, sodium chloride, magnesium sulphate, calcium chloride, sucrose and sterile water.

In some embodiments, the compositions further comprise at least one adjuvant or other substance useful for enhancing an immune response. For instance, the invention includes a pharmaceutical composition comprising a live, attenuated Salmonella bacterium of the invention with a CpG oligodeoxynucleotide adjuvant. Adjuvants with a CpG motif are described, for instance, in US Patent Application 20060019239, which is herein incorporated by reference in its entirety.

Other suitable adjuvants that may be used in a pharmaceutical composition with the attenuated microorganism of the invention, include, but are not limited to, aluminium salts such as aluminium hydroxide, aluminum oxide and aluminium phosphate, oil-based adjuvants such as Freund's Complete Adjuvant and Freund's Incomplete Adjuvant, mycolate-based adjuvants (e.g., trehalose dimycolate), bacterial lipopolysaccharide (LPS), peptidoglycans (e.g., mureins, mucopeptides, or glycoproteins such as N-Opaca, muramyl dipeptide [MDP], or MDP analogs), proteoglycans (e.g., extracted from Klebsiella pneumoniae), streptococcal preparations (e.g., OK432), muramyldipeptides, Immune Stimulating Complexes (the “Iscoms” as disclosed in EP 109 942, EP 180 564 and EP 231 039), saponins, DEAE-dextran, neutral oils (such as miglyol), vegetable oils (such as arachis oil), liposomes, polyols, the Ribi adjuvant system (see, for example, GB-A-2 189 141), vitamin E, Carbopol or interleukins, particularly those that stimulate cell mediated immunity.

In other embodiments, the compositions may comprise a carrier useful for protecting the attenuated microorganism from the stomach acid or other chemicals, such as chlorine from tap water, that may be present at the time of administration. For example, the microorganism may be administered as a suspension in a solution containing sodium bicarbonate and ascorbic acid (plus aspartame as sweetener).

Suitable formulations for oral administration include hard or soft gelatin capsules, pills, tablets, including coated tablets, elixirs, suspensions, syrups or inhalations and controlled release forms thereof. Gelatin capsules can serve as carriers for lyophilized pharmaceutical compositions.

Suitable formulations also include compositions for administered to a subject by way of a pharmaceutical implant. Methods of making pharmaceutical implants are known in the art and include, for instance, implants such as those disclosed in US patent application 2005/0202072, which is herein incorporated by reference in its entirety. Pharmaceutical implants may be inserted in close proximity to the site of an infection or at a location suitable for eliciting an immune response for the prevention or treatment of of an infection. In another embodiment, the pharmaceutical implant is inserted in a tumor or in close proximity to a tumor for the prevention or treatment of a cancer.

The compositions of the present invention can be administered via parenteral, topical, intratumoral, subcutaneous, intravenous, intramuscular, intraperitoneal, transdermal and buccal routes. Alternatively, or concurrently, administration may be noninvasive by either the oral, intratumoral, inhalation, nasal, or pulmonary route.

Suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension, such as sodium carboxymethyl cellulose, sorbitol and dextran. Optionally, the suspension may also contain stabilizers. Liposomes can also be used to encapsulate the agent for delivery into the cell.

In certain embodiments, the pharmaceutical composition dosage is 1.0×10⁵ to 1.0×10¹⁵ CFU/ml or cells/ml. For instance, the invention includes a pharmaceutical composition with about 1.0×10⁵, 1.5×10⁵, 1.0×10⁶, 1.5×10⁶, 1.0×10⁷, 1.5×10⁷, 1.0×10⁸, 1.5×10⁸, 1.0×10⁹, 1.5×10⁹, 1.0×10¹⁰, 1.5×10¹⁰, 1.0×10¹¹, 1.5 ×10¹¹, 1.0×10¹², 1.5×10¹², 1.0×10¹³, 1.5×10¹³, 1.0×10¹⁴, 1.5×10¹⁴ or about 1.0×10¹⁵ CFU/ml or cells/ml. For instance, the invention includes a pharmaceutical composition dosage of about 1.0×10⁸ to about 1.0×10¹⁰ CFU/ml or cells/ml. In some embodiments, the dosage administered will be dependent upon the age, health, and weight of the recipient, type of concurrent treatment, if any, frequency of treatment, and the nature of the effect desired.

The compositions of the invention may be co-administered along with other compounds typically prescribed for the prevention or treatment of an infection by the pathogen or related condition according to generally accepted medical practice.

The present invention also provides a method for immunizing a subject against a pathogen. In one embodiment, the method comprises administering a pharmaceutical composition comprising an attenuated microorganism, wherein the attenuated microorganism expresses a fusion protein, said fusion protein comprising an amino acid sequence from E. coli CS3 protein consisting essentially of an export signal fused to at least one heterologous antigen amino acid sequence from the pathogen. The pharmaceutical composition may be administered orally to a subject. The subject may be at risk for acquiring an infection from the pathogen, may be suffering from an infection from the pathogen, or may have a recurrent infection caused by the pathogen. Accordingly, the present invention includes methods of preventing and treating pathogen infections by administering a composition comprising the attenuated microorganisms described herein that express fusion proteins containing heterologous antigens from the pathogens. In some embodiments, the pathogen is enterotoxigenic E. coli (ETEC). The fusion proteins expressed by the attenuated microorganisms may comprise amino acid sequences from the heat stable and subunits from the heat labile enterotoxins of ETEC bacteria.

In some embodiments, the method of the invention induces an effective immune response to the pathogen in the subject. As used herein, the term “effective immune response” refers to an immune response that confers protective immunity. For instance, an immune response can be considered to be an “effective immune response” if it is sufficient to prevent a subject from developing an infection from the pathogen after administration of a challenge dose of the pathogen or administration of pathogenic toxins. An effective immune response may comprise a humoral immune response and/or a cell mediated immune response. In one embodiment, the effective immune response refers to the ability of the pharmaceutical composition of the invention to elicit the production of antibodies. An effective immune response may give rise to mucosal immunity. See, for instance, Holmgren and Czerkinsky, Nature Medicine, Vol. 11:S45-S53 (2005). In a preferred embodiment, the composition induces mucosal immunity against the pathogen in the subject.

In some embodiments, the method of the invention may reduce the incidence of (or probability of) recurrent infection by the pathogen, such as ETEC. In other embodiments, the composition of the invention is administered to a subject post-infection, thereby ameliorating the symptoms and/or course of the illness caused by the pathogen, as well as preventing recurrence.

The pharmaceutical composition may be administered to the subject once, or may be administered a plurality of times, such as one, two, three, four or five times. If the composition is administered a plurality of times, it may be administered at regular intervals, e.g., monthly, or may be staged as with pulsed dosing.

The present invention also encompasses methods of generating antibodies to a heterologous antigen in a subject. In one embodiment, the method comprises administering to a subject an isolated recombinant fusion protein as described herein. The recombinant fusion protein may comprise an amino acid sequence from the heterologous antigen fused to an amino acid sequence from E. coli CS3 protein containing an export signal. In another embodiment, the method comprises expressing a nucleic acid encoding a recombinant fusion protein as described herein in an attenuated microorganism, such as an attenuated Salmonella, and administering the attenuated microorganism to the subject. Preferably, the attenuated microorganism (e.g. attenuated Salmonella) expressing the recombinant fusion protein displays the recombinant fusion protein on its surface or secretes the fusion protein. In some embodiments, the recombinant fusion protein may be cleaved by the attenuated microorganism prior to the export and/or secretion of the recombinant fusion protein. The antibodies generated in the subject according to the methods of the invention may, in some embodiments, provide short term immunity against a pathogen from which the heterologous antigen was derived.

The antibodies generated by the methods described above may be isolated and purified from the subject. Accordingly, the present invention also encompasses isolated antibodies to the recombinant fusion proteins of the invention as well as antibodies to attenuated microorganisms (e.g. attenuated Salmonella) expressing the recombinant fusion proteins on their surface. Methods of making and purifying polyclonal antibodies are known to those of skill in the art. For example, the polyclonal antibodies may be isolated from the subject's serum after one or more administrations of the recombinant fusion protein or an attenuated microorganism expressing the recombinant fusion protein. Alternatively, monoclonal antibodies to the recombinant fusion proteins may be produced by generating hybridoma cultures expressing the recombinant fusion protein. See, for example, Harlow and Lane, Antibodies: A Laboratory Manual (New York: Cold Spring Harbor Laboratory Press, 1988).

The present invention also encompasses methods of treating pathogenic infections or diseases in a subject using the constructs and attenuated microorganisms of the invention. In some embodiments, the method comprises administering to a subject an attenuated microorganism comprising a nucleic acid encoding a CS3 peptide and a heterologous antigen from a pathogen to treat or prevent an infection caused by the pathogen. In other embodiments, the method comprises administering to a subject an attenuated microorganism comprising a nucleic acid encoding a CS3 peptide and a cancer antigen to treat or prevent various forms of cancer. In other embodiments the method comprises administering to a subject an attenuated microorganism comprising a nucleic acid encoding a CS3 peptide and a therapeutic protein, for example, a cytokine, to treat or prevent various forms of cancer.

In one embodiment of the invention, an attenuated microorganism comprising a nucleic acid encoding a CS3 signal peptide and an enterotoxigenic E. coli (ETEC) antigen is administered to a subject to prevent or treat an ETEC infection. An ETEC antigen may include an ETEC toxin such as heat stable toxin (ST) and heat labile toxin (LT) and non toxic subunits and detoxified mutants thereof, aerobactin, hemolysin, fimbriae and colonization factor antigens (e.g., CFA/I, CFA/II, CFA/IVPCFO159, PCFO166, CS4, CS5, CS6, CS7 and CS17).

ETEC is an important cause of diarrhea in infants and travelers in underdeveloped countries or regions of poor sanitation. In the U.S., it has been implicated in sporadic waterborne outbreaks, as well as due to the consumption of soft cheeses, Mexican-style foods and raw vegetables. The diseases vary from minor discomfort to a severe cholera-like syndrome. ETEC is acquired by ingestion of contaminated food and water, and adults in endemic areas typically develop immunity. The disease requires colonization and elaboration of one or more enterotoxins. The illness is self-limited; the diarrhea usually lasts fewer than 5 days. Because the duration of illness is short, ETEC infections generally do not require antibiotic therapy. Treatment is mainly supportive including oral or intravenous fluids for rehydration. Occasionally antibiotics, such as ciprofloxacin for adults and trimethoprim/sulfamethoxazole for children, are given if the patient has an underlying illness or if the diarrhea is severe. ETEC infection may cause dehydration but there are generally no serious complications or long-term sequelae from this infection.

In one embodiment of the invention, an attenuated microorganism comprising a nucleic acid encoding a CS3 signal peptide and ETEC antigen (e.g., ST and/or LT and immunogenic portions thereof) is administered to a subject to prevent or ameliorate symptoms associated with an ETEC infection. In another embodiment, the attenuated microorganism is a traveler's vaccine. In yet another embodiment, the traveler's vaccine comprises one or more types of attenuated microorganisms, each microorganism expressing one or more heterologous antigens from a gastrointestinal pathogen (e.g., one or more heterologous antigens from E. coli types such as ETEC, Enteropathogenic E. coli (EPEC), Enterohaemorrhagic E. coli (EHEC) and/or Enteroinvasive E. coli (EIEC); Salmonella spp. such as S. typhi and S. typhimurium; Shigella spp. such as S. dysenteriae and S. flexneri; Clostridium spp. such as C. difficile and C. botulinum; Vibrio spp. such as V. cholera, Listeria spp. such as L. monocytogenes; Campylobacter spp. such as C. jejuni and/or Rotavirus).

EPEC is a significant cause of diarrhea world-wide, with disease occurring most frequently in developing countries. In these countries, disease occurs regularly in hospitals and clinics, as well as in the general community. EPEC outbreaks in developed countries, on the other hand, usually consist of sporadic, isolated incidents which are localized to neonatal nurseries of hospitals or day-care centers. Infants less than 6 months of age are most often affected, although EPEC is also capable of causing disease in children and adults. EPEC is a cause of diarrheal morbidity and mortality among children in developing countries. Clinical symptoms of EPEC infection in children consist of diarrhea which varies in duration (days to months) and severity. In addition to profuse watery stool, symptoms include dehydration, fever, vomiting and weight loss. In protracted or severe cases, disease is often associated with the delayed growth of children, metabolic acidosis (decrease in blood pH resulting from a loss of bicarbonate) and, in extreme cases, death.

In one embodiment of the invention, an attenuated microorganism comprising a nucleic acid encoding a CS3 signal peptide and an EPEC antigen is administered to a subject to prevent an EPEC infection. For instance, in one embodiment, the attenuated microorganism is a traveler's vaccine. An EPEC antigen may include EPEC EspA, EspB, EspD, intimin, Tir, and flagellin proteins. An attenuated microorganism comprising a nucleic acid encoding a CS3 signal peptide and an EPEC antigen may be administered to a subject to prevent a condition or symptoms associated with an EPEC infection. In some embodiments, the attenuated microorganism of the invention is administered to a subject to treat an EPEC infection (i.e., provided post-exposure). In these embodiments, an attenuated microorganism comprising a nucleic acid encoding a CS3 signal peptide and EPEC antigen is administered to a subject to reduce the severity of an EPEC infection (i.e., ameliorate the symptoms associated with an EPEC infection).

The invention also includes a method of administering to a subject an attenuated microorganism to prevent or treat an enterohemorrhagic E. coli (EHEC) invention. The attenuated microorganism may be an attenuated Salmonella spp. cell comprising a nucleic acid encoding a CS3 signal peptide and EHEC antigen. Such an attenuated microorganism may be capable of expressing one or more EHEC antigens known in the art, including, but not limited to, verotoxin 1 (shiga-like toxin 1), verotoxin 2, EspA, EspB, EspD, Tir, intimin, and nucleolin.

EHEC serotype 0157:H7 is a Shiga-like toxin (Stx)-producing E. coli (STEC). 0157:H7 infection resembles shigellosis and is most severe in children, the elderly and immunocompromised subjects. It the most frequent cause of acute renal failure in children in the US. In addition to renal failure, conditions associated with an EHEC infection include, but are not limited to, hemorrhagic colitis and hemolytic uremic syndrome (HUS). HUS, a condition frequently associated with the young who become infected with 0157-H7, is characterized by renal failure and hemolytic anemia. The disease can lead to permanent loss of kidney function. Elderly who become infected with 0157-H7 are at risk for thrombotic thrombocytopenic purpura (TTP), a condition characterized by HUS, fever and neurologic symptoms. The mortality rate in the elderly is as high as about 50%.

There is no evidence that antibiotics improve the course of disease, and it is thought that treatment with some antibiotics may precipitate kidney complications. Blood transfusions and kidney dialysis are often required for those who develop HUS. A lengthy hospital stay is usually necessary. With intensive hospital care and treatment, death occurs in 3-5% of HUS cases. Long-term complications of about one third of HUS cases include abnormal kidney function requiring long-term dialysis. Approximately 8% of remaining patients have additional complications, including high blood pressure, seizures, blindness, paralysis, and partial bowel removal.

In one embodiment of the invention, an attenuated microorganism comprising a nucleic acid encoding a CS3 signal peptide and an EHEC antigen is administered to a subject to prevent an EHEC infection. For instance, in one embodiment, the attenuated microorganism is a traveler's vaccine. In another embodiment, an attenuated microorganism comprising a nucleic acid encoding a CS3 signal peptide and EHEC antigen is administered to a subject to prevent a condition or symptoms associated with an EHEC infection such as hemorrhagic colitis, kidney failure, HUS and TTP. For instance, the invention includes vaccinating a subject at risk for developing an EHEC infection (e.g. the young, immunocompromised, and elderly) against EHEC infections, such as an O157-H7 infection, by administering to the subject an attenuated microorganism comprising a nucleic acid encoding a CS3 signal peptide and an EHEC antigen. The attenuated microorganism of the invention can also be administered to a subject to treat an EHEC infection (i.e., provided post-exposure). In this embodiment, an attenuated microorganism comprising a nucleic acid encoding a CS3 signal peptide and EHEC antigen is administered to a subject to reduce the severity of an EHEC infection.

Enteroinvasive E. coli (EIEC) closely resemble Shigella in their pathogenic mechanisms and the kind of clinical illness they produce. EIEC penetrate and multiply within epithelial cells of the colon causing widespread cell destruction. The clinical syndrome is identical to Shigella dysentery and includes a dysentery-like diarrhea with fever.

In one embodiment of the invention, an attenuated microorganism comprising a nucleic acid encoding a CS3 signal peptide and an EIEC antigen is administered to a subject to prevent an EIEC infection. EIEC antigens include, but are not limited to, adhesins and Ipa proteins (e.g., IpaA, IpaB, IpaC, IpaD and IpaH). In another embodiment, an attenuated microorganism comprising a nucleic acid encoding a CS3 signal peptide and an EIEC antigen is administered to a subject to prevent a condition or symptoms associated with EIEC infection.

Enteroaggregative E. coli (EAggEC) is associated with persistent diarrhea, especially in children in the developing world. Persistent diarrhea lasts more than 14 days and is a major cause of illness and death. EAggEC is also a leading cause of traveler's diarrhea.

In one embodiment of the invention, an attenuated microorganism comprising a nucleic acid encoding a CS3 signal peptide and an EAggEC antigen is administered to a subject to prevent an EAggEC infection. In another embodiment, an attenuated microorganism comprising a nucleic acid encoding a CS3 signal peptide and an EAggEC antigen is administered to a subject to prevent a condition or symptoms associated with EAggEC infection, for instance, persistent diarrhea. The attenuated microorganism of the invention can also be administered to a subject to treat an EAggEC infection (i.e., provided post-exposure). In this embodiment, an attenuated microorganism comprising a nucleic acid encoding a CS3 signal peptide and an EAggEC antigen is administered to a subject to reduce the severity of the infection (i.e., ameliorate symptoms of an infection). EAggEC antigens useful for the methods of the invention include, but are not limited to, hemolysin and ST enterotoxin.

In one embodiment of the invention, the method comprises administering to a subject an attenuated microorganism comprising a nucleic acid encoding a CS3 signal peptide and a C. difficile antigen to prevent a C. difficile infection. For instance, in one embodiment, the attenuated organism is administered to prevent the reoccurrence of a C. difficile infection. Clostridium difficile is a major cause of nosocomial diarrhea in industrialized countries. C. difficile is a commensal bacterium of the human intestine in a minority of the population. Patients who have been staying long-term in a hospital or a nursing home have a higher likelihood of being colonized by this bacterium. In small numbers it does not result in disease of any significance. Antibiotics, especially those with a broad spectrum of activity, cause disruption of normal intestinal flora, leading to an overgrowth of C. difficile, which flourishes under these conditions. Although many cases respond to available therapy; infection can increase morbidity, prolong hospitalization, and produce life-threatening colitis. There are also major problems with infection recurrence after the initial episode.

In another embodiment of the invention, an attenuated microorganism comprising a nucleic acid encoding a CS3 signal peptide and a C. difficile antigen is administered to a subject to prevent a condition or symptoms associated with C. difficile, for instance, pseudomembranous colitis. The attenuated microorganism of the invention can also be administered to a subject to treat a C. difficile infection (i.e., provided post-exposure). In this embodiment, an attenuated microorganism comprising a nucleic acid encoding a CS3 signal peptide and a C. difficile antigen is administered to a subject to reduce the severity of the infection (i.e., ameliorate symptoms of an infection).

The pathogenesis of C. difficile-associated diarrhea (CDAD) is mediated by the actions of two large protein exotoxins, toxin A and toxin B, which induce mucosal injury and inflammation of the colon. For instance, in one embodiment of the invention, the C. difficile antigen comprises a C. difficile antigen disclosed in U.S. provisional application 61/086,673, filed Aug. 6, 2008, which is herein incorporated by reference in its entirety. In another embodiment, the C. difficile antigen comprises common antigen (i.e., glutamate dehydrogenase) or an immunogenic fragment thereof.

Chlamydia spp. infect a variety of host species and are associated with a wide range of different disease pathologies, including genital, ocular and neonatal infection. C. trachomatis is the world's most common cause of sexually transmitted disease. The World Health Organization estimates that at least 90 million people are infected each year. In the United States, genital infection with C. trachomatis is the single most frequently reported infectious disease, with an estimated 4 million cases per year. Approximately 10% of women suffering C. trachomatis genital infection eventually become infertile. Children born to C. trachomatis-infected mothers are at high risk of ocular and respiratory infection. C. trachomatis is also a leading cause of ocular infection in tropical and sub-tropical nations, causing blindness in an estimated 6 million people per year. (Schachter, Julius, Chapter 6, “Infection and Disease Epidemiology,” pp 139-169., in Chlamydia: Intracellular Biology, Pathogenesis and Immunity, Stephens, Richard S., ed., ASM Press, Washington, DC, 1999.; Hogan, et al., Infect Immun., 72(4):1843-555 (2004); Peipert, N Engl J Med, 349:2424-30 (2003)).

In one embodiment of the invention, the method comprises administering to a subject an attenuated microorganism comprising a nucleic acid encoding a CS3 signal peptide and a C. trachomatis antigen to prevent a C. trachomatis infection. In another embodiment, an attenuated microorganism comprising a nucleic acid encoding a CS3 signal peptide and a C. trachomatis antigen is administered to a subject to prevent a condition or symptoms associated with C. trachomatis, for instance, prostatitis, urethritis, epididymitis, cervicitis, pelvic inflammatory disease, pelvic pain, newborn eye infection, newborn lung infection, infertility, proctitis, reactive arthritis and trachoma. The attenuated microorganism of the invention can also be administered to a subject to treat a C. trachomatis infection (i.e., provided post-exposure). In this embodiment, an attenuated microorganism comprising a nucleic acid encoding a CS3 signal peptide and a C. trachomatis antigen is administered to a subject to reduce the severity of the infection (i.e., ameliorate symptoms of an infection). C. trachomatis antigens that can be used with the constructs and methods of the invention include, for instance, Chlamydial PmpG, PmpD, PmpE, PmpI, HtrA, OmcD and/or OmpH polypeptide or an immunogenic fragment thereof. In one embodiment of the invention, the Chlamydial polypeptide lacks an N terminal domain. In another embodiment, the Chlamydial polypeptide lacks a transmembrane domain. In one embodiment of the invention, the Chlamydial polypeptide is a PmpG CT84 and/or CT110 polypeptide as disclosed in PCT application PCT/US2008/007490, filed Jun. 16, 2008, which is herein incorporated by reference in its entirety. In another embodiment, the Chlamydial antigen or antigens is disclosed in PCT application PCT/US2008/06656, filed May 23, 2008, which is herein incorporated by reference in its entirety.

Chlamydia pneumoniae, which commonly causes asymptomatic acute respiratory tract infection and pneumonia followed by lifelong chronic infection, has been particularly associated with incipient and advanced atherosclerosis. C. pneumoniae is also associated with coronary artery disease, myocardial infarction, carotid artery disease, cerebrovascular disease, coronary heart disease, carotid artery stenosis, aortic aneurysm, claudication and stroke.

The invention encompasses a method for treating or preventing a C. pneumoniae infection in a subject. In one embodiment, the method comprises administering to a subject an attenuated microorganism comprising a nucleic acid encoding a CS3 signal peptide and a C. pneumoniae antigen to prevent a C. pneumoniae infection. In another embodiment, an attenuated microorganism comprising a nucleic acid encoding a CS3 signal peptide and a C. pneumoniae antigen is administered to a subject to prevent a condition or symptoms associated with C. pneumoniae, for instance, asymptomatic acute respiratory tract infection, pneumonia, atherosclerosis, coronary artery disease, myocardial infarction, carotid artery disease, cerebrovascular disease, coronary heart disease, carotid artery stenosis, aortic aneurysm, claudication and stroke. The attenuated microorganism of the invention can also be administered to a subject to treat a C. pneumoniae infection (i.e., provided post-exposure). In this embodiment, an attenuated microorganism comprising a nucleic acid encoding a CS3 signal peptide and a C. pneumoniae antigen is administered to a subject to reduce the severity of the infection (i.e., ameliorate symptoms of an infection). In one embodiment of the invention, the C. pneumoniae antigen (or antigens) comprises a Chlamydial PmpG, PmpD, PmpE, PmpI, HtrA, OmcD and/or OmpH polypeptide or an immunogenic fragment thereof. In another embodiment, the Chlamydial polypeptide lacks an N terminus domain. In another embodiment, the Chlamydial polypeptide lacks a transmembrane domain. In one embodiment of the invention, the Chlamydial polypeptide is a PmpG CT84 and/or CT110 polypeptide as disclosed in PCT application PCT/US2008/007490, filed Jun. 16, 2008, which is herein incorporated by reference in its entirety. In another embodiment, the Chlamydial antigen or antigens is disclosed in PCT application PCT/US2008/06656, filed May 23, 2008, which is herein incorporated by reference in its entirety.

In another embodiment, the present invention provides a method for preventing a cholera infection comprising administering to a subject an attenuated microorganism comprising a nucleic acid encoding a CS3 signal peptide and a V. cholera antigen. V. cholerae antigens include, for instance, cholera toxin, cholera toxin A subunit, cholera toxin B subunit or a toxin-coregulated pilus protein (e.g., TcpA, TcpB, TcpC, TcpD, TcpE and TcpF). V. cholerae produces cholera toxin, the model for enterotoxins, whose action on the mucosal epithelium is responsible for the characteristic diarrhea of the disease cholera. The clinical description of cholera begins with sudden onset of massive diarrhea. The patient may lose gallons of protein-free fluid and associated electrolytes, bicarbonates and ions within a day or two. This results from the activity of the cholera enterotoxin, which activates the adenylate cyclase enzyme in intestinal cells, converting the cells into pumps that extract water and electrolytes from blood and tissues and pump them into the lumen of the intestine. This loss of fluid leads to dehydration, anuria, acidosis and shock. The loss of potassium ions may result in cardiac complications and circulatory failure. Untreated cholera frequently results in high (50-60%) mortality rates.

Thus, in another embodiment of the invention, an attenuated microorganism comprising a nucleic acid encoding a CS3 signal peptide and a V. cholerae antigen is administered to a subject to prevent a condition or symptoms associated with cholera. The attenuated microorganism of the invention can also be administered to a subject to treat a cholera infection (i.e., provided post-exposure). In this embodiment, an attenuated microorganism comprising a nucleic acid encoding a CS3 signal peptide and a V. cholerae antigen is administered to a subject to reduce the severity of the infection (i.e., ameliorate symptoms of an infection).

P. mirabilis, a Gram-negative bacterium, is a frequent cause of complicated urinary tract infections in those with functional or anatomical abnormalities or those subject to long-term catheterization. In one embodiment of the invention, an attenuated microorganism comprising a nucleic acid encoding a CS3 signal peptide and a P. mirabilis antigen is administered to a subject to prevent a P. mirabilis infection. In another embodiment, an attenuated microorganism comprising a nucleic acid encoding a CS3 signal peptide and a P. mirabilis antigen is administered to a subject to prevent a condition or symptoms associated with P. mirabilis, for instance, rheumatoid arthritis. The attenuated microorganism of the invention can also be administered to a subject to treat a P. mirabilis infection (i.e., provided post-exposure). In this embodiment, an attenuated microorganism comprising a nucleic acid encoding a CS3 signal peptide and a P. mirabilis antigen is administered to a subject to reduce the severity of the infection. A Proteus antigen may be a P. mirabilis antigen, such as PMI0842 and PMI2596 (see, Nielubowicz, G. R. et al., Infect. Immun. 76(9):4222-4231 (2008), which discloses P. mirabilis vaccine candidate antigens and is herein incorporated by reference in its entirety). The invention also includes as antigens Proteus iron acquisition proteins and immunogenic fragments thereof.

Listeriosis, a serious infection caused by eating food contaminated with the bacterium Listeria monocytogenes, has recently been recognized as an important public health problem in the United States. The disease affects primarily persons of advanced age, pregnant women, newborns, and adults with weakened immune systems. A person with listeriosis has fever, muscle aches, and sometimes gastrointestinal symptoms such as nausea or diarrhea. If infection spreads to the nervous system, symptoms such as headache, stiff neck, confusion, loss of balance, or convulsions can occur. Infected pregnant women may experience only a mild, flu-like illness. However, infections during pregnancy can lead to miscarriage or stillbirth, premature delivery, or infection of the newborn.

In one embodiment of the invention, an attenuated microorganism comprising a nucleic acid encoding a CS3 signal peptide and a Listeria antigen is administered to a subject to prevent listeriosis . In another embodiment, an attenuated microorganism comprising a nucleic acid encoding a CS3 signal peptide and a Listeria antigen is administered to a subject to prevent a condition or symptoms associated with listeriosis. Listeria antigens that can be used with the constructs and methods of the invention include, for instance, Listeria listeriolysin or p60 as well as immunogenic fragments thereof

Hepatitis B virus infection may either be acute (self-limiting) or chronic (long-standing). Persons with self-limiting infection clear the infection spontaneously within weeks to months. Acute infection with hepatitis B virus is associated with acute viral hepatitis—an illness that begins with general ill-health, loss of appetite, nausea, vomiting, body aches, mild fever, dark urine, and then progresses to development of jaundice. It has been noted that itchy skin has been an indication as a possible symptom of all hepatitis virus types. The illness lasts for a few weeks and then gradually improves in most affected people. A few patients may have more severe liver disease (fulminant hepatic failure), and may die as a result of it. The infection may be entirely asymptomatic and may go unrecognized. Chronic infection with Hepatitis B virus may be either asymptomatic or may be associated with a chronic inflammation of the liver (chronic hepatitis), leading to cirrhosis over a period of several years. This type of infection dramatically increases the incidence of hepatocellular carcinoma (liver cancer). Hepatitis B virus has also been linked to the development of Membranous glomerulonephritis (MGN).

Hepatitis C is a blood-borne infectious disease that is caused by the Hepatitis C virus (HCV), affecting the liver. The infection is often asymptomatic, but once established, chronic infection can cause inflammation of the liver (chronic hepatitis) as well as scarring of the liver (fibrosis and cirrhosis). In some cases, those with cirrhosis (advanced scarring) will go on to develop liver failure or other complications of cirrhosis, including liver cancer.

In one embodiment of the invention, an attenuated microorganism comprising a nucleic acid encoding a CS3 signal peptide and a hepatitis B or C antigen is administered to a subject to prevent a hepatitis B or C infection, respectively, and/or liver cancer. Hepatitis B viral antigens, include, but are not limited to, hepatitis B surface antigen and hepatitis B core antigen. Hepatitis C viral antigens, include, but are not limited to hepatitis C core antigen.

In another embodiment, an attenuated microorganism comprising a nucleic acid encoding a CS3 signal peptide and a hepatitis antigen is administered to a subject to prevent a condition or symptoms associated with hepatitis such as fibrosis or cirrhosis of the liver or liver cancer. The attenuated microorganism of the invention can also be administered to a subject to treat a hepatitis B or C infection (i.e., provided post-exposure) and/or liver cancer. In this embodiment, an attenuated microorganism comprising a nucleic acid encoding a CS3 signal peptide and a hepatitis antigen is administered to a subject to reduce the severity of the infection, including, for instance, to reduce the severity of liver damage or risk of development of liver cancer. In one embodiment, the attenuated microorganism is administered to limit infection, i.e., prevent chronic hepatitis infection.

In another embodiment, the present invention provides a method for preventing HIV infection comprising administering to a subject an attenuated microorganism comprising a nucleic acid encoding a CS3 signal peptide and HIV antigen. HIV antigens include, but are not limited to, p24, gp120, Tat, rev and pol. In another embodiment, an attenuated microorganism comprising a nucleic acid encoding a CS3 signal peptide and an HIV antigen is administered to a subject to prevent a condition or symptoms associated with HIV. In this embodiment, an attenuated microorganism comprising a nucleic acid encoding a CS3 signal peptide and an HIV antigen is administered to a subject to reduce the severity of the infection.

The present invention includes a method of treating HPV infection comprising administering to a subject an attenuated microorganism comprising a nucleic acid encoding a CS3 signal peptide and an HPV antigen. HPV antigens include, but not limited to, E6 and E7 peptides. In one embodiment the HPV antigen(s) is an HPV antigen from an HPV type associated with cervical cancer such as HPV types 16, 18, 31, 33, 35, 39, 45, 51, 52, 56, 58, 59 or 68. In another embodiment, the HPV antigen(s) comprise antigens from HPV types 6, 11, 16 and 18. In another embodiment, an attenuated microorganism comprising a nucleic acid encoding a CS3 signal peptide and an HPV antigen is administered to a subject to prevent a condition or symptoms associated with HPV. In this embodiment, an attenuated microorganism comprising a nucleic acid encoding a CS3 signal peptide and an HPV antigen is administered to a subject to reduce the severity of the infection.

The present invention includes an attenuated microorganism comprising a nucleic acid encoding a CS3 signal peptide and Herpesvirus antigen. In one embodiment, an attenuated microorganism comprising a nucleic acid encoding a CS3 signal peptide and a Herpesvirus antigen is administered to a subject to prevent a Herpesvirus infection. A Herpesvirus antigen may include a Herpes Simplex virus antigen, such as thymidine kinase or a fragment thereof.

In another embodiment, an attenuated microorganism comprising a nucleic acid encoding a CS3 signal peptide and a Herpesvirus antigen is administered to a subject to prevent a condition or symptoms associated with Herpesvirus. In this embodiment, an attenuated microorganism comprising a nucleic acid encoding a CS3 signal peptide and a Herpesvirus antigen is administered to a subject to reduce the severity of the infection.

In another embodiment of the invention, an attenuated microorganism comprising a nucleic acid encoding a CS3 signal peptide and a Rotavirus antigen(s) is administered to a subject to prevent a Rotavirus infection. For instance, in one embodiment, the attenuated microorganism is travelers vaccine or a component of a travelers vaccine. In another embodiment, the attenuated microorganism is a childhood vaccine (i.e., administered to children) for the prevention of a Rotavirus infection. An attenuated microorganism comprising a nucleic acid encoding a CS3 signal peptide and a Rotavirus antigen may also be administered to a subject to prevent a condition(s) or symptom(s) associated with Rotavirus. The attenuated microorganism of the invention can also be administered to a subject to treat Rotavirus infection (i.e., provided post-exposure). In this embodiment, an attenuated microorganism comprising a nucleic acid encoding a CS3 signal peptide and a Rotavirus antigen is administered to a subject to reduce the severity of the infection. A Rotavirus antigen may include antigens VP4, VP6, and VP7.

Seasonal influenza is a respiratory illness that can be transmitted person to person. Pandemic influenza, on the other hand, is a global outbreak of disease that occurs when a new influenza A virus appears in humans, causes serious illness and then spreads easily from person to person worldwide. Three major influenza pandemics swept the globe in the 20th century causing millions of deaths, and no one knows for sure when the next pandemic may strike. Avian influenza H5N1 is deadly to domestic fowl, can be transmitted from birds to humans, and is deadly to humans. There is virtually no human immunity, and human vaccine availability is very limited. If H5N1 virus were to gain the capacity to spread easily from person to person, a pandemic could begin.

Thus, the present invention includes a method to prevent an influenza infection (e.g., seasonal flu or pandemic flu) comprising administering to a subject an attenuated microorganism comprising a nucleic acid encoding a CS3 signal peptide and an influenza antigen. In one embodiment, an attenuated microorganism comprising a nucleic acid encoding a CS3 signal peptide and an influenza antigen is administered to a subject to prevent a condition or symptoms associated with influenza. In this embodiment, an attenuated microorganism comprising a nucleic acid encoding a CS3 signal peptide and an influenza antigen is administered to a subject to reduce the severity of the infection. Influenza antigens include, but are not limited to, hemagglutinin (HA), neuraminidase (NA) and nucleoprotein.

Malaria is a vector-borne infectious disease caused by protozoan parasites Plasmodium falciparum, Plasmodium vivax, Plasmodium ovale and Plasmodium malariae. It is widespread in tropical and subtropical regions, including parts of the Americas, Asia, and Africa. Each year, there are approximately 515 million cases of malaria, killing between one and three million people, the majority of whom are young children in Sub-Saharan Africa.

In one embodiment of the invention, an attenuated microorganism comprising a nucleic acid encoding a CS3 signal peptide and a Plasmodium antigen is administered to a subject to prevent a malaria infection. In another embodiment, an attenuated microorganism comprising a nucleic acid encoding a CS3 signal peptide and a Plasmodium antigen is administered to a subject to prevent a condition or symptoms associated with malaria. The attenuated microorganism of the invention can also be administered to a subject to treat a malaria infection (i.e., provided post-exposure). In this embodiment, an attenuated microorganism comprising a nucleic acid encoding a CS3 signal peptide and a Plasmodium antigen is administered to a subject to reduce the severity of the infection (i.e., ameliorate symptoms of an infection). Malarial antigens that can be used with the genetic constructs and methods of the invention include, for instance, apical membrane antigen 1 (AMA-1), acidic basic repeat antigen (ABRA or p101), gametocyte antigen 11.1, circumsporozoite protein 1 (CSP-1), erythrocyte binding proteins, P. falciparum erythrocyte membrane protein 1 (PfEMP-1), glutamate-rich protein (GLURP), heat shock proteins, histidine-rich protein 2 (HRP-2), knob-associated histidine-rich protein (KAHRP), mature-parasite-infected erythrocyte membrane antigen (MESA/PfEMP-2), merozoite surface protein 1 (MSP-1), merozoite surface protein 2 (MSP-2), ring-infected erythrocyte surface antigen (RESA/Pf155), rhoptry associated protein (RAP), serine repeat antigen (SERA), S antigen and Pf332 protein.

The present invention provides a method for treating cancer. In one embodiment, the method comprises administering to a subject an attenuated microorganism comprising a nucleic acid encoding a CS3 signal peptide and alpha-fetoprotein (AFP) or fragment thereof. High blood levels of AFP are common with hepatocellular carcinoma (HCC), germ cell tumors (e.g., tumors of the testes and ovaries) and metastatic cancer of the liver (i.e., cancer originating in other organ). The serum AFT level of patients is considered to be attributable to the expression of AFP in liver cell carcinoma, and the AFP antigen is thought to be controlled through liver cancer cell-specific expression. Studies focused on the immunological treatment of liver cancer using this liver cancer-specific antigen have been conducted. See, for instance, WO 08/072895, which is herein incorporated by reference in its entirety, In one embodiment of the invention, an attenuated microorganism comprising a nucleic acid encoding a CS3 signal peptide and AFP or fragment thereof is administered to a subject at risk for liver cancer (e.g., a subject suffering from hepatitis B, hepatitis C or HIV) for the prevention of liver cancer. In another embodiment of the invention, an attenuated microorganism comprising a nucleic acid encoding a CS3 signal peptide and AFP or a fragment thereof is administered to a subject for the treatment of a liver cancer (e.g., HCC) or germ cell tumor cancer. Suitable alpha-fetoprotein peptides for use in the methods of the invention include synthetic fragments such as Growth Inhibitory Peptide (GIP) and those disclosed in U.S. Pat. No. 6,818,741, which is herein incorporated by reference in its entirety.

AFP has also been found to interfere with estrogen-dependent responses, including the growth-promoting effects of estrogen on breast cancer. It has been demonstrated that AFP purified from a human hepatoma culture and then injected into tumor-bearing immune-deficient mice stops the growth of estrogen-receptor-positive (ER+) but not estrogen-receptor-negative (ER−) human breast cancer xenographs. See, J. A. et al., Clin. Cancer Res. 4:2877-2884 (1998), which is herein incorporated by reference in its entirety. Accordingly, it is envisioned that an attenuated microorganism comprising a nucleic acid encoding a CS3 signal peptide and AFP or a fragment thereof can be administered to subject for the prevention or treatment of breast cancer. It is also envisioned that an attenuated microorganism comprising a nucleic acid encoding a CS3 signal peptide and AFP or a fragment thereof can be administered to a subject for the prevention or treatment of an ER+ cancer. In one embodiment, chemotherapy such as tamoxifen is administered concurrently with the attenuated microorganism.

The invention also includes methods for the prevention or treatment of cancer comprising administering to a subject an attenuated microorganism comprising a nucleic acid encoding a CS3 signal sequence and one or more cancer-testis (CT) antigens. The CT family of antigens is composed of antigens expressed solely in the testicular germ cells of normal adult males and in various cancer cells. Members ofthis family, include, but are not limited to, MAGE (e.g., MAGE-A1, MAGE-A2, MAGE-A3, MAGE-A4, MAGE-A5, MAGE-A6, MAGE-A8, MAGE-A9, MAGE-A10, MAGE-A11, MAGE-A12, MAGE-B1, MAGE-B2, MAGE-B5, MAGE-B6, MAGE-C1, MAGE-C2, MAGE-C3, MAGE-D4, MAGE-E1); BAGE (e.g., BAGE, BAGE2, BAGE3, BAGE4 and BAGE5); LAGE-1; GAGE (e.g., GAGE1, GAGE2a, GAGE3, GAGE4, GAGES, GAGE6, GAGE7 and GAGE8); SSX (e.g., SSX1, SSX2, SSX2b, SSX3 and SSX4); SYCP1; BRDT; SPANX (e.g., SPANXA1, SPANXA2, SPANXB1, SPANXB2, SPANXC, SPANXD, SPANXE, SPANXN1, SPANXN2, SPANXN3, SPANXN4, SPANXN5); XAGE (e.g., XAGE1, XAGE1B, XAGE1C, XAGE1D, XAGE1E, XAGE2, XAGE2B, XAGE3, XAGE-3b, XAGE-4/RP 11-167P23.2 and XAGE5); CT45; CT47; PAGE-5 (e.g., PAGE1, PAGE2, PAGE2b, PAGE3 and PAGE4); CTAGE-1; TSPY-1 (e.g., TSPY2, TSPY1D, TSPY1E, TSPY1F, TSPY1G, TSPY1H and TSPY1I) and NY-ESO-1 (e.g., CTAG1A, CTAG1B, CTAG2, LAGE-1b). Although NY-ESO-1 antigens have been noted as being perhaps the most immunogenic of this family and promising as cancer vaccine antigens, other CT family members such as MAGE antigens and LAGE-1 have been suggested for use and/or tested in clinical trials for use as cancer vaccine antigens. The invention includes an attenuated microorganism comprising a nucleic acid encoding a CS3 signal sequence and one or more CT antigens.

NY-ESO-1, LAGE-1 and MAGE proteins (e.g., MAGE-A antigen such as MAGE-A3 and MAGE-A4) and peptide fragments thereof have been proposed for use or have been used in clinical trials for the vaccination of patients with breast cancer (see, for instance, Sugita, Y. et al., Cancer Research, 64: 2199-2204 (2004); Ludwig Institute for Cancer Research Phase 1 Clinical Trial NCT00299728); non-small cell lung carcinoma (see, for instance, Yoshida, N. et al., Int. J. Oncol., 28(5):1089-1098 (2006); Ludwig Institute for Cancer Research Phase 1 Clinical Trial NCT00299728); serous ovarian neoplasms (see, for instance, Yakirevich, E. et al., Clin. Cancer Res., 9(17):6453-6460 2003)); malignant melanomas (Adams, S. et al., J. Immunol., 181(1):776-784 (2008); Ludwig Institute for Cancer Research Phase 1 Clinical Trial NCT00299728); hepatocellular carcinoma (Ludwig Institute for Cancer Research Phase 1 Clinical Trial NCT00299728); bladder cancer (Sharma, P. et al., Cancer Immunity, 3(19) (2003); Ludwig Institute for Cancer Research Phase 1 Clinical Trial NCT00299728); sarcoma (Ludwig Institute for Cancer Research Phase 1 Clinical Trial NCT00299728); prostate cancer (Ludwig Institute for Cancer Research Phase 1 Clinical Trial NCT00299728); esophageal cancer (Ludwig Institute for Cancer Research Phase 1 Clinical Trial NCT00299728); thyroid cancer (Maio, M. et al., J. Clin. Endocrinol. Metab., 88(2):748-754 (2003); and ovarian cancer (Odunsi, K. et al., PNAS, 104(31):12837-12842 (2007); Odunsi, K., et al., Cancer Res., 63(18):6076-6083 (2003); Ludwig Institute for Cancer Research Phase 1 Clinical Trial NCT00299728).

In one embodiment of the invention, a MAGE (e.g., MAGE-A antigen such as MAGE-A3 MAGE-A4), LAGE-1 and/or NY-ESO-1 antigen is expressed on the surface of an attenuated microorganism by way of a CS3 signal peptide. Accordingly, it is envisioned that au attenuated microorganism comprising a nucleic acid encoding a CS3 signal peptide and, a MAGE protein, LAGE-1 protein and/or NY-ESO-1 protein or a fragment thereof can be administered to a subject for the prevention or treatment of a cancer. In one embodiment, the attenuated microorganism capable of expressing a MAGE, LAGE-1 and/or NY-ESO-1 antigen is administered to a subject for the prevention (including prevention of reoccurrence) or treatment of a cancer selected from the group consisting of breast cancer, skin cancer, liver cancer, esophageal cancer, prostate cancer, bladder cancer, lung cancer, thyroid cancer and ovarian cancer. In one embodiment, the IMAGE protein is selected from the group consisting of MAGE-A1, MAGE-A2, MAGE-A3, MAGE-A4, MAGE-A5, MAGE-A6, MAGE-A8, MAGE-A9, MAGE-A10, MAGE-A11, MAGE-A12, MAGE-B1, MAGE-B2, MAGE-B5, MAGE-B6, MAGE-C1, MAGE-C2, MAGE-C3, MAGE-D4, MAGE-E1. In another embodiment, the MAGE protein is a MAGE-A protein. The attenuated microorganism may be administered in combination with chemotherapy. Further, the attenuated microorganism may be administered before and after surgical procedures to remove cancerous tissue.

MAGE antigens, in particular MAGEC1/CT7 and MAGEA3/6, and LAGE-1 have also been suggested as good candidates for immunotherapy for treatment of multiple myeloma (see, for instance, Andrade, V. C. et al., Cancer Immun. 8(2) (2008)). Accordingly, it is envisioned that an attenuated microorganism of the invention expressing a MAGE and/or LAGE-1 antigen can be used for the prevention or treatment of multiple myeloma. In one embodiment, the invention includes a method of preventing (including preventing reoccurrence) or treating multiple myeloma comprising administering an attenuated microorganism comprising a nucleic acid encoding a CS3 signal peptide and an MAGE antigen (e.g., MAGE-C1 or MAGE-A3) and/or a LAGE-1 antigen to a subject.

Cancer antigen 125 (CA-125) is a cancer antigen that is over-expressed by some ovarian cancer cells and breast cancer cells as well other cancerous cell types. In one embodiment, the invention includes a method for preventing or treating cancer (including ovarian cancer or breast cancer) comprising administering to a subject an attenuated microorganism comprising a nucleic acid encoding a CS3 signal peptide and CA-125. In another embodiment, the cancer is a CA-125 over-expressing cancer, for instance, CA-125 over-expressing ovarian cancer or CA-125 over-expressing breast cancer. The attenuated microorganism may be administered in combination with chemotherapy. In one embodiment, the attenuated microorganism is administered before and after surgical procedures to remove cancerous tissue such as ovarian tissue or breast tissue.

Cancer antigen 15-3, also known as MUC-1, is a cancer antigen that is over-expressed in some cancers, for instance, some breast cancers. In one embodiment, the invention includes an attenuated microorganism comprising a nucleic acid encoding a CS3 signal peptide and cancer antigen 15-3. In another embodiment, an attenuated microorganism comprising a nucleic acid encoding a CS3 signal peptide and cancer antigen 15-3 is administered to a subject for the prevention or treatment of cancer, including, but not limited to, breast cancer. In another embodiment, the cancer is a cancer antigen 15-3 over-expressing cancer, for instance, cancer antigen 15-3 over-expressing breast cancer. The attenuated microorganism may be administered in combination with chemotherapy. In one embodiment, the attenuated microorganism is administered before and after surgical procedures to remove cancerous tissue such as cancerous breast tissue.

CD20 has an important functional role in B cell activation, proliferation, and differentiation. CD20 has been a natural focus for monoclonal antibody therapy because of its relatively high degree of expression in B cell malignancies, perhaps as high as 95% in follicular lymphomas. The present invention includes an attenuated microorganism comprising a nucleic acid encoding a CS3 signal peptide and CD80 or an immunogenic fragment thereof. In one embodiment, the attenuated microorganism expressing CD80 or an immunogenic fragment thereof is administered to a subject for the prevention or treatment of a B cell malignancy. For instance, the attenuated microorganism expressing CD80 or an immunogenic fragment thereof can be administered to a subject for the prevention (including prevention of reoccurrence) or treatment of non-Hodgkin lymphoma. The invention includes the administration of an attenuated microorganism expressing CD80 or a fragment thereof in conjunction with conventional chemotherapy, including, but not limited to, cyclophosphamide, doxorubicin, vincristine, and prednisone.

Cell-surface glycoprotein 17-1A is expressed on epithelial tissues and on various carcinomas. Edrecolomab (MAb17-1A, Panorex®) is a chimeric mouse/human monoclonal antibody to 17-1A. Preliminary studies had shown promise of a possible use in patients with stage III colorectal carcinoma (with metastatis to the lymph nodes). Edrecolomab was well tolerated and studies are underway to determine whether it can be of use in the treatment of other forms of cancer.

The present invention also includes an attenuated microorganism comprising a nucleic acid encoding a CS3 signal peptide and 17-1A. In one embodiment, the attenuated microorganism capable of expressing 17-1A is administered to a subject for the prevention or treatment of cancer, including, but not limited to, colon cancer (e.g., advanced colorectal carcinoma). The attenuated microorganism may be administered in combination with chemotherapy. In one embodiment, the attenuated microorganism is administered before and after surgical procedures to remove cancerous tissue.

HER-2/neu has been reported as being over-expressed in some breast, ovarian and stomach cancers. In particular, the HER-2/neu antigen is over-expressed in 25% to 35% of breast cancers A HER-2/neu monoclonal antibody, Trastuzumab (Herceptin®), has been developed for the treatment of HER-2/neu+ breast cancers. It is believed that the antibody works in a variety of ways, including, for instance, through downregulation of HER-2 receptor expression, inhibition of proliferation of human tumor cells that over-express HER-2 protein, enhancement of immune recruitment and antibody-dependent cell-mediated cytotoxicity against tumor cells that over-express HER-2 protein, and downregulation of angiogenesis factors. In phase I and II trials of patients with metastatic breast cancer, treatment with a combination of trastuzumab and cisplatin resulted in prolongation of survival and higher response rates than that seen with cisplatin alone.

Thus, in one embodiment of the invention, an attenuated microorganism comprising a nucleic acid encoding a CS3 signal peptide and HER-2/neu is administered to a subject for the prevention or treatment of cancer, including, but not limited to breast cancer, ovarian cancer and stomach cancer. In another embodiment, the cancer is HER-2/neu over-expressing cancer, for instance, HER-2/neu over-expressing breast cancer. The attenuated microorganism may be administered in combination with chemotherapy. In another embodiment, the attenuated microorganism is administered before and after surgical procedures to remove cancerous tissue.

Carcinoembryonic antigen (CEA) is found in high levels on tumors in people with colorectal, lung, breast and pancreatic cancer as compared with normal tissue. CEA is thought to be released into the bloodstream by tumors. Patients have been shown to mount T-cell responses to CEA. The present invention also encompasses administration of an attenuated microorganism comprising a nucleic acid encoding a CS3 signal peptide and CEA to a subject for the prevention or treatment of cancer, including, but not limited to colorectal, lung, breast and pancreatic cancer. In one embodiment, the tumor cells of the cancer over-express CEA. The attenuated microorganism may be administered in combination with chemotherapy. In one embodiment, the attenuated microorganism is administered before and after surgical procedures to remove cancerous tissue.

MART-1 (also known as Melan-A) is an antigen expressed by melanocytes. It is a specific melanoma cancer marker that is recognized by T cells and is more abundant on melanoma cells than normal cells. Tyrosinase is a key enzyme involved in the initial stages of melanin production. Studies have shown that tyrosinase is a specific marker for melanoma and is more abundant on melanoma cells than normal cells.

In another embodiment, the present invention provides a method of preventing or treating skin cancer (e.g. melanoma) comprising administering to a subject an attenuated microorganism comprising a nucleic acid encoding a CS3 signal peptide and MART-1 and/or tyrosinase or an immunogenic fragment thereof. In one embodiment, the tumor cells of the cancer over-express MART-1 and/or tyrosinase. The attenuated microorganism may be administered in combination with chemotherapy. In one embodiment, the attenuated microorganism is administered before and after surgical procedures to remove cancerous tissue.

Prostate Specific Antigen (PSA) is a prostate-specific protein antigen that can be found circulating in the blood, as well as on prostate cancer cells. PSA generally is present in small amounts in men who do not have cancer, but the quantity of PSA generally rises when prostate cancer develops. The higher a man's PSA level, the more likely it is that cancer is present, but there are many other possible reasons for an elevated PSA level. Patients have been shown to mount T-cell responses to PSA.

In one embodiment, the invention includes an attenuated microorganism comprising a nucleic acid encoding a CS3 signal peptide and PSA. In another embodiment, an attenuated microorganism comprising a nucleic acid encoding a CS3 signal peptide and PSA is administered to a subject for the prevention or treatment of cancer, including, but not limited to prostate cancer. In another embodiment, the tumor cells of the cancer over-express PSA. The attenuated microorganism may be administered in combination with chemotherapy. In one embodiment, the attenuated microorganism is administered before and after surgical procedures to remove cancerous tissue, such as cancerous prostate tissue.

IL-2 and interferon-α 2b are two cytokines approved by the FDA for treatment of cancer. IL-2 has demonstrated efficacy against renal cell cancer, melanoma, lymphoma, and leukemia. Interferon has demonstrated similar efficacy, but also in Kaposi's sarcoma, chronic myelogenous leukemia and hairy cell leukemia. Overall, cytokines are substances that appear to have application in the treatment of hematologic malignancies or immunogenic tumors.

Other cytokines like interferon-β (IFN-β), Tumor Necrosis Factor-α (TNF-α), Tumor Necrosis Factor-β (TNF-β), IL-1, 4, 6, 12, 15 and the Colony Stimulating Factors (CFSs) have shown a certain antitumoral activity on some types of tumors and therefore are the object of further studies. For instance, TNF-α is administered locally to treat advanced soft tissue sarcomas as well as metastatic melanoma. (See, for instance, Horssen, R. V. et al., The Oncologist, 11:397-408 (2006). Granulocyte-monocyte colony stimulating factor (GM-CSF) is a well-known cytokine, approved for use in stem cell and bone marrow transplant to reconstitute the myeloid series. GM-CSF is a protein that stimulates the proliferation of antigen-presenting cells.

In one embodiment, the present invention provides a method of delivering a therapeutic peptide to a subject comprising administering to the subject an attenuated microorganism comprising a nucleic acid encoding a CS3 signal peptide and a therapeutic peptide. Theraputic peptides suitable for use in the methods of the invention include, but are not limited to, cytokines, such as IL-1, IL-2, IL-4, IL-6, IL-12, IL-15, IFN-α, IFN-β, TNF-α, TNF-β, and GM-CSF. In one embodiment, an attenuated microorganism comprising a nucleic acid encoding a CS3 signal peptide and a cytokine is administered to a subject for the prevention or treatment of cancer. In another embodiment, the attenuated microorganism may expresse a cytokine and a cancer antigen. In another embodiment, an attenuated microorganism comprising a nucleic acid encoding a CS3 signal peptide and a cytokine is co-administered to a subject with an attenuated microorganism expressing a cancer antigen In yet another embodiment, an attenuated microorganism comprising a nucleic acid encoding a CS3 peptide and an interleukin is administered as an adjuvant. Further, the attenuated microorganism may be administered in combination with chemotherapy. In another embodiment, the attenuated microorganism is administered before and after surgical procedures to remove cancerous tissue.

The examples which follow are set forth to illustrate the present invention, and are not to be construed as limiting thereof.

### EXAMPLES

### Example 1

### Design of CS3 Fusion Constructs

Although the following example describes CS3 fusion constructs containing antigens from enterotoxigenic E. coli (ETEC), it is to be understood that any heterologous antigen can be used with the CS3 export signal sequence in the fusion construct.

### CS3 Signal Peptide:Ltb:ST Fusion

A first construct is created containing the export signal sequence from the ETEC surface antigen, CS3. The nucleotide and amino acid sequence of the export signal from CS3 antigen is shown below:

### Nucleotide Sequence of CS3 Export Signal Sequence:

(SEQ ID NO: 7)
atgttaaaaataaaatacttattaataggtctttcactgtcagctatgagttcatactcactagct

### Amino Acid Sequence of CS3 Export Signal Sequence:

(SEQ ID NO: 8)
MLKIKYLLIGLSLSAMSSYSLA

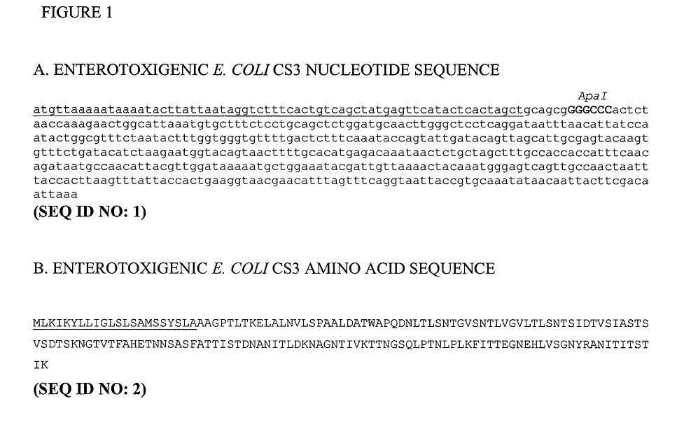

The export signal sequence is isolated from the full length CS3 sequence using the natural restriction site for ApaI (See underlined nucleotides in FIG. 1A). The CS3 export signal sequence is placed under the control of a Salmonella ssaG promoter using standard molecular biology techniques. Cleavage of the CS3 protein at the ApaI site provides a four amino acid linker (AAGP) with which the sequence encoding the heterologous antigen is fused to the CS3 export signal sequence.

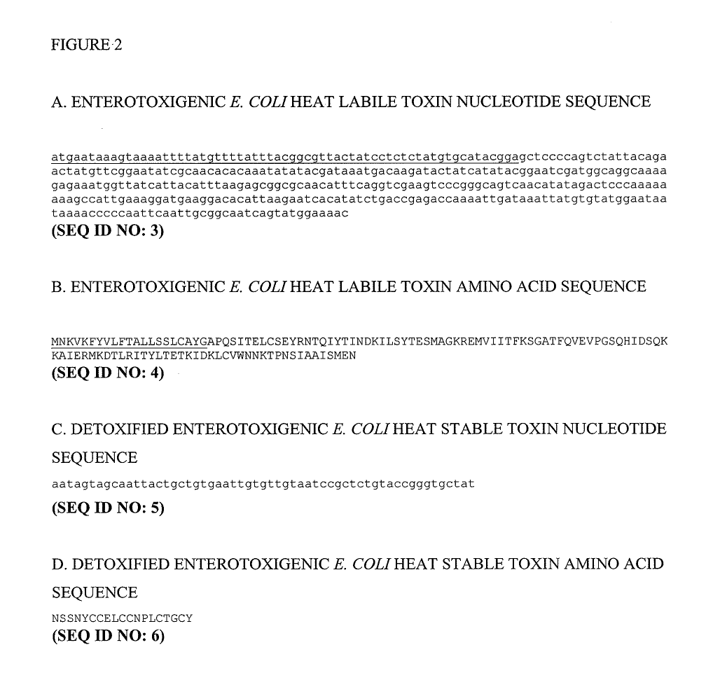

A detoxified mutant of ETEC heat stable toxin (ST) and a two amino acid residue linker (PG) is inserted by inverse PCR into a nucleotide sequence encoding the B subunit of the heat labile toxin from ETEC (LTb) The full length nucleotide and amino acid sequences for ST and LTb are shown in FIG. 2. The ST open reading frame is followed by a sequence to introduce stop codons in all reading frames. The proline-glycine linker is co-amplified to give a flexible connection. The inverse PCR primers are as follows (the homologous region is shaded and the sequence of the stable toxin is underlined):

### LTB ST Forward

(SEQ ID NO: 9)

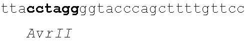

### LTB ST Reverse

(SEQ ID NO: 10)

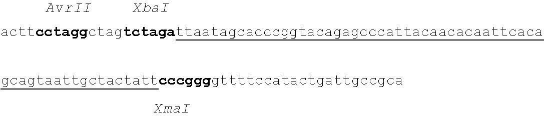

ST is added by the reverse primer. After amplification of the plasmid with these primers, the PCR products are digested with AvrII and ligated at this sticky end to form a LTb:ST plasmid. The polynucleotide encoding the LtB:ST can then be inserted into the first construct described above to create an in-frame genetic fusion with the CS3 export signal sequence under the control of a Salmonella ssaG promoter. The nucleotide and amino acid sequence of the resulting fusion protein is shown below. The underlined region denotes the CS3 export signal sequence, while the shaded sequence in capital letters shows the linker residues and restriction enzyme sites. The sequence encoding the proline-glycine linker residues is a restriction site for SmaI. The amino acid sequence for LTb is shown in italics.

### Nucleotide Sequence of CS3 (Export Signal):LTb:ST Fusion

(SEQ ID NO: 11)
atgttaaaaataaaatacttattaataggtctttcactgtcagctatgagttcatactcactagctgc

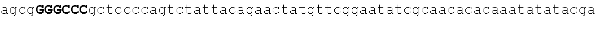

taaatgacaagatactatcatatacggaatcgatggcaggcaaaagagaaatggttatcattacattt
aagagcggcgcaacatttcaggtcgaagtcccgggcagtcaacatatagactcccaaaaaaaagccat
tgaaaggatgaaggacacattaagaatcacatatctgaccgagaccaaaattgataaattatgtgtat

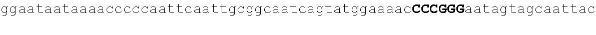

tgctgtgaattgtgttgtaatccgctctgtaccgggtgctattaa

### Amino Acid Sequence of CS3 (Export Signal):LTb:ST Fusion

(SEQ ID NO: 12)

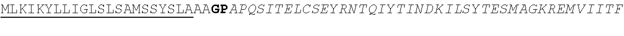

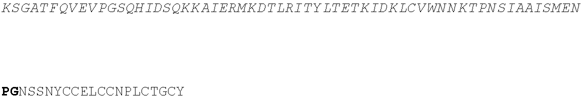

### CS3 (Full Length):Ltb:ST Fusion

A second construct is created using the full length CS3 protein as an export system for heterologous antigens. The nucleotide and amino acid sequence for the full length CS3 protein is shown in FIGS. 1A and 1B, respectively. In this construct, the nucleotide sequence encoding the full length CS3 protein is placed under the control of a Salmonella ssaG promoter. The nucleotide sequence encoding the Ltb:ST fusion antigen described above is cloned in-frame with the full length CS3 protein to generate an amino terminal fusion of the CS3 protein to the Ltb:ST fusion antigen. The nucleotide and amino acid sequences of the resulting fusion protein are shown below. The underlining indicates the CS3 export signal sequence and the shaded sequence in capital letters shows the linker residues and restriction sites. The LTb sequence is again depicted in italics in the amino acid sequence.

### Nucleotide Sequence of CS3 (Full Length):LTb:ST Fusion

(SEQ ID NO: 13)
atgttaaaaataaaatacttattaataggtctttcactgtcagctatgagttcatactcactagctgc
agcggggcccactctaaccaaagaactggcattaaatgtgctttctcctgcagctctggatgcaactt
gggctcctcaggataatttaacattatccaatactggcgtttctaatactttggtgggtgttttgact
ctttcaaataccagtattgatacagttagcattgcgagtacaagtgtttctgatacatctaagaatgg
tacagtaacttttgcacatgagacaaataactctgctagctttgccaccaccatttcaacagataatg
ccaacattacgttggataaaaatgctggaaatacgattgttaaaactacaaatgggagtcagttgcca
actaatttaccacttaagtttattaccactgaaggtaacgaacatttagtttcaggtaattaccgtgc

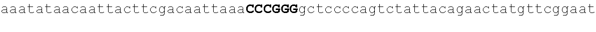

atcgcaacacacaaatatatacgataaatgacaagatactatcatatacggaatcgatggcaggcaaa
agagaaatggttatcattacatttaagagcggcgcaacatttcaggtcgaagtcccgggcagtcaaca
tatagactcccaaaaaaaagccattgaaaggatgaaggacacattaagaatcacatatctgaccgaga
ccaaaattgataaattatgtgtatggaataataaaacccccaattcaattgcggcaatcagtatggaa

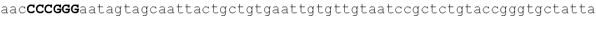

a

### Amino Acid Sequence of CS3 (Full Length):LTb:ST Fusion

(SEQ ID NO: 14)
MLKIKYLLIGLSLSAMSSYSLAAAGPTLTKELALNVLSPAALDATWAPQDNLTLSNTGVSNTLVGVLT
LSNTSIDTVSIASTSVSDTSKNGTVTFAHETNNSASFATTISTDNANITLDKNAGNTIVKTTNGSQLP

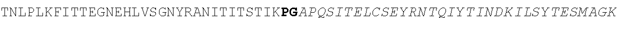

REMVIITFKSGATFQVEVPGSQHIDSQKKAIERMKDTLRITYLTETKIDKLCVWNNKTPNSIAAISME

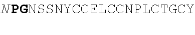

### Example 2

### Construct Containing C. difficile Toxin B Full Length CS3 Fusion

E. coli codon optimized crdB will be isolated from pPCRscript FAFB as a Sma I/Avr II fragment and cloned into the corresponding sites in the pMBS CS3 ST vector, thereby creating a CS3 crdB fusion protein. Following confirmation of the structure of this vector the promoter CS3 crdB fusion will then be isolated as a Xho I/Avr II fragment and sub cloned into a suicide vector based on pCVD442. This suicide vector will have been previously engineered to contain DNA sequence derived from the intended integration site in the S. typhi genome, which will have been modified to include Xho I and Avr II sites at the insertion site, as in, for example, the pCVDaro or pCVDssaV suicide vector.

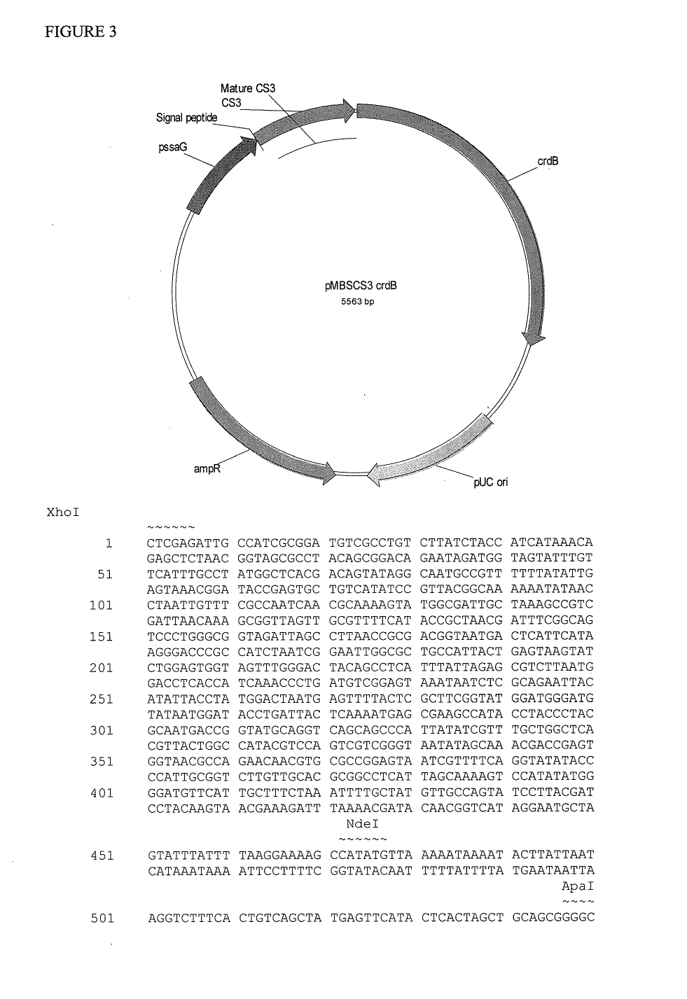

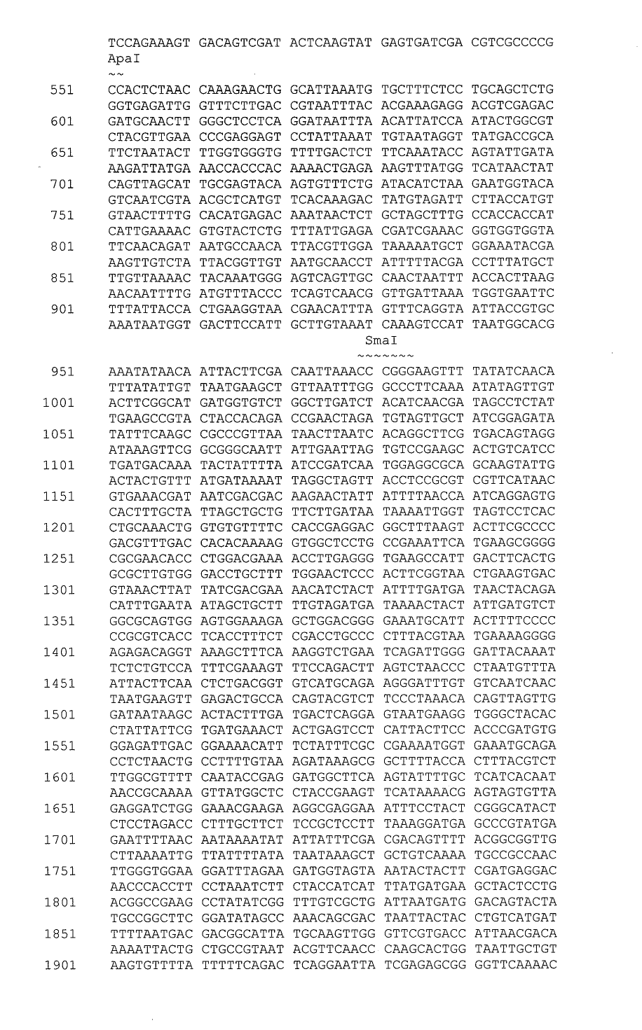

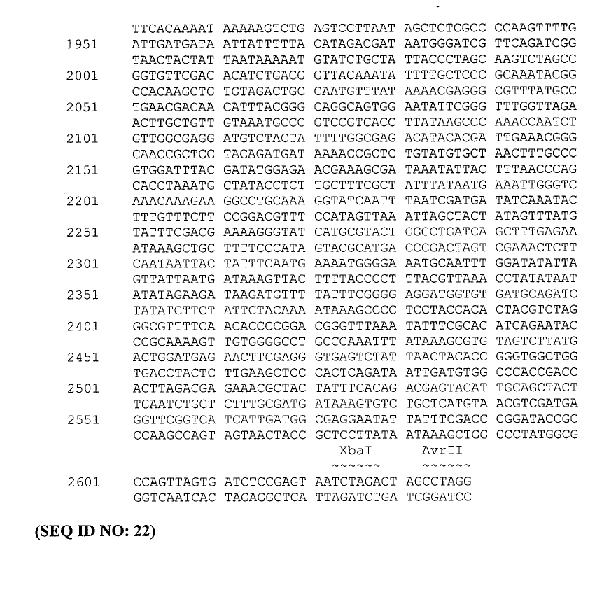

Suicide vectors derived from pCD442 contain the R6k origin of replication which requires the lambda phage pir protein to allow replication in plasmid form. Following transfer of the plasmid into Salmonella by chemical or elctro transformation or conjugation from E. coli, the amplicilin marker gene present on the plasmid will only be retained if the plasmid is integrated into the Salmonella chromosome by homologous recombination. Following selection of clones containing the integrated plasmid (merodiploids) these clones will be cultured in the absence of antibiotics to allow a second round of recombination to occur, clones having lost the plasmid will then be selected by plating onto media containing sucrose. The plasmid encoded sacB gene encodes levan sucrase activity, conversion of sucrose to levan is toxic to gram negative organisms. The second recombination step will result in clones which have either reverted to their pre transformed state or clones containing the integrated promoter antigen fusion. Clones containing the antigen expression cassette would then be identified by PCR. The structure of the construct is depicted in FIG. 3 and SEQ ID NO. 22.

### Example 3

### Construct Containing C. difficile Toxin B CS3 Signal Peptide Fusion

In order to construct the promoter CS3 signal peptide crdB fusion, E. coli codon optimized crdB fusion from pPCRscript FAFB will be amplified by PCR amplification using primers designed to introduce an Apa I restriction endonuclease recognition site at the 5′ end of the crdB sequence and retaining the Avr II site present at the 3′ end. The PCR product will then be cloned between the Apa I/Avr II sites present in the pMBS CS3 ST vector. Following confirmation of the structure of this vector, the promoter CS3 crdB fusion will then be isolated as a Xho I/Avr II fragment and sub cloned into a suicide vector based on pCVD442. This suicide vector will have been previously engineered to contain a DNA sequence derived from the intended integration site in the S. typhi genome, which will have been modified to include Xho I and Avr II sites at the insertion site, for example this suicide vector could be pCVDaro or pCVDssaV.

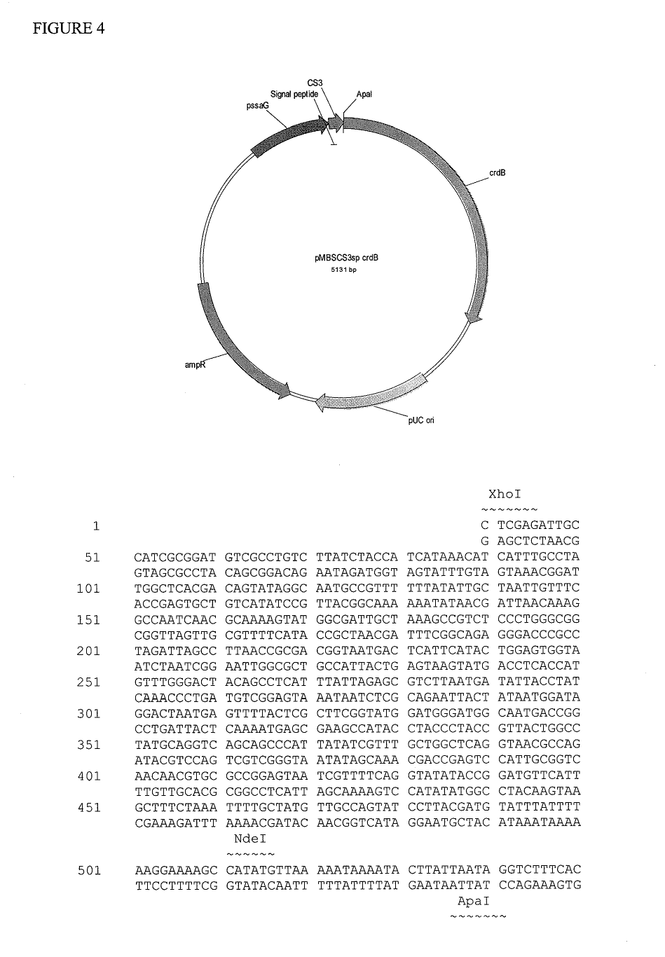

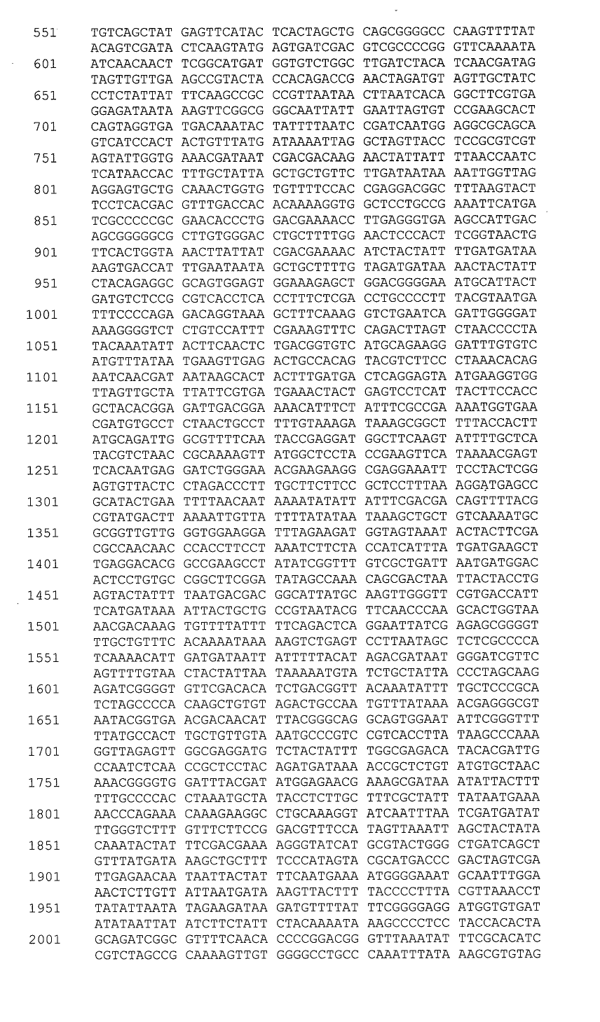

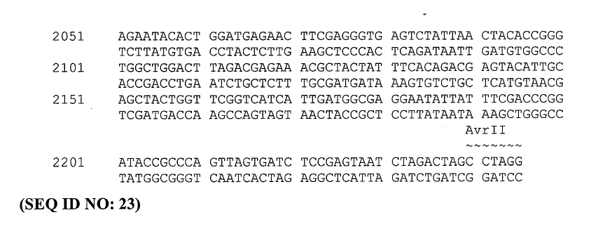

Suicide vectors derived from pCD442 contain the R6k origin of replication which requires the lambda phage pir protein to allow replication in plasmid form. Following transfer of the plasmid into Salmonella by chemical or elctro transformation or conjugation from E. coli, the amplicilin marker gene present on the plasmid will only be retained if the plasmid is integrated into the Salmonella chromosome by homologous recombination. Following selection of clones containing the integrated plasmid (merodiploids) these clones will be cultured in the absence of antibiotics to allow a second round of recombination to occur, clones having lost the plasmid will then be selected by plating onto media containing sucrose. The plasmid encoded sacB gene encodes levan sucrase activity, conversion of sucrose to levan is toxic to gram negative organisms. The second recombination step will result in clones which have either reverted to their pre transformed state or clones containing the integrated promoter antigen fusion, Clones containing the antigen expression cassette would then be identified by PCR. The structure of the construct is depicted in FIG. 4 and SEQ ID NO. 23.

### Example 4

### Construct Containing C. difficile Toxin A Full Length CS3 Fusion

In order to construct the promoter CS3 signal peptide crdA fusion, E. coli codon optimized crdA fusion from pPCRscript FAFB will be amplified by PCR amplification using primers designed to introduce an Sma I restriction endonuclease recognition site at the 5′ end of the crdA sequence and retaining the Avr II site present at the 3′ end. The PCR product will then be cloned between the Sma I/Avr II sites present in the pMBS CS3 ST vector. Following confirmation of the structure of this vector, the promoter CS3 crdA fusion will then be isolated as a Xho I/Avr II fragment and subcloned into a suicide vector based on pCVD442. This suicide vector will have been previously engineered to contain a DNA sequence derived from the intended integration site in the S. typhi genome, which will have been modified to include Xho I and Avr II sites at the insertion site, for example this suicide vector could be pCVDaro or pCVDssaV.

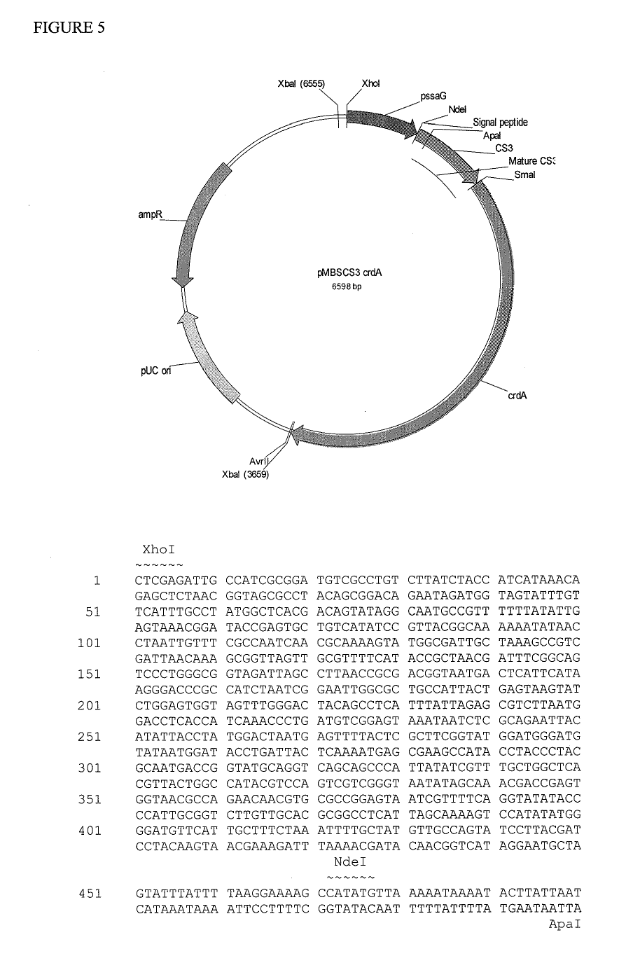

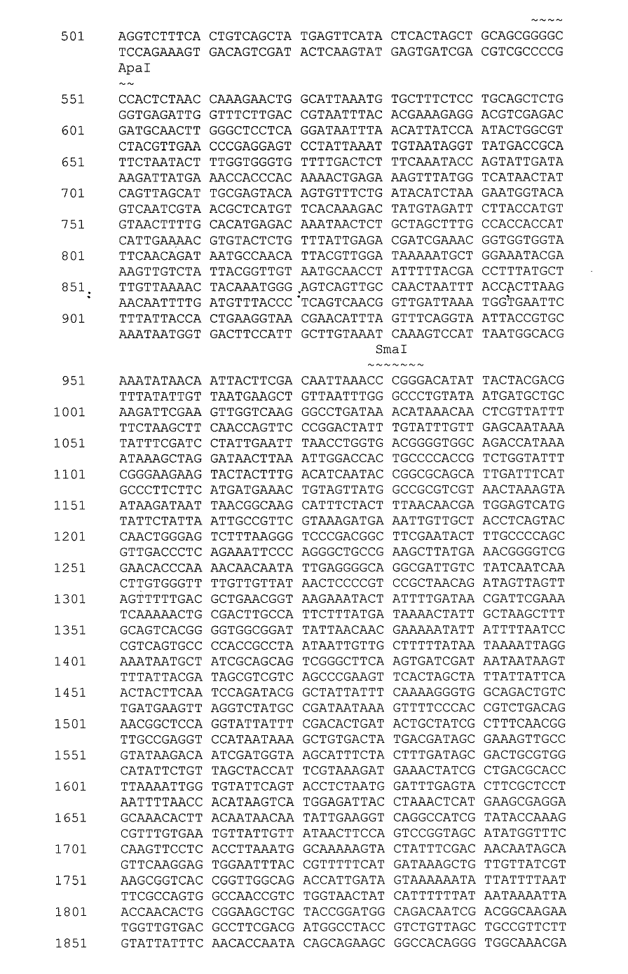

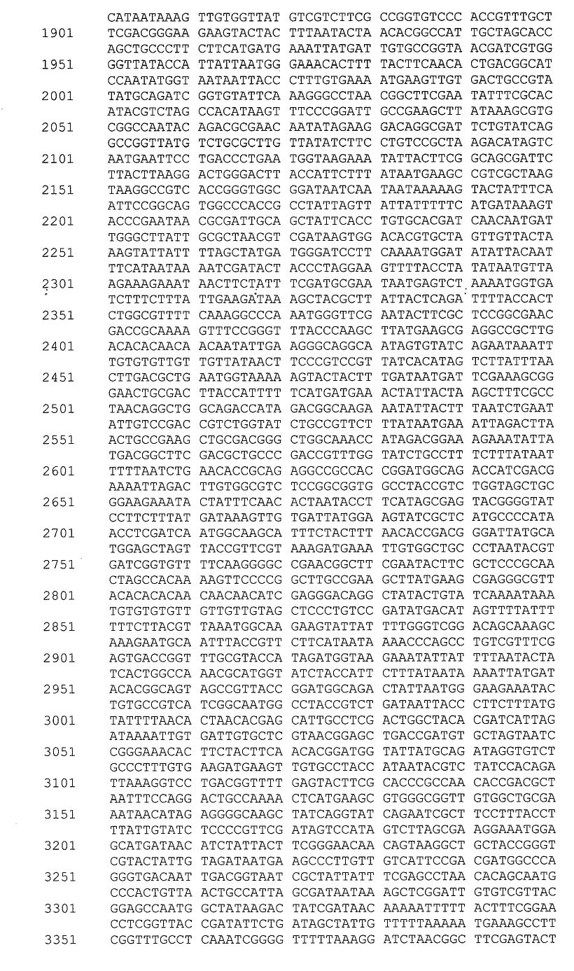

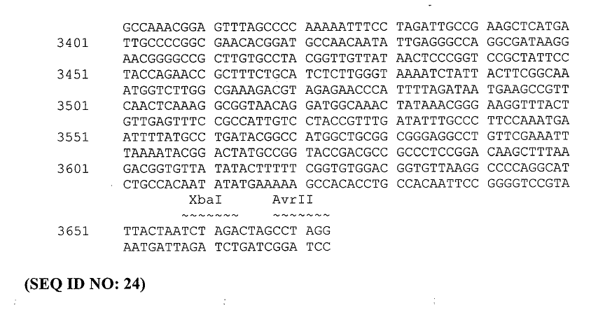

Suicide vectors derived from pCD442 contain the R6k origin of replication which requires the lambda phage pir protein to allow replication in plasmid form. Following transfer of the plasmid into Salmonella by chemical or elctro transformation or conjugation from E. coli, the amplicilin marker gene present on the plasmid will only be retained if the plasmid is integrated into the Salmonella chromosome by homologous recombination. Following selection of clones containing the integrated plasmid (merodiploids) these clones will be cultured in the absence of antibiotics to allow a second round of recombination to occur, clones having lost the plasmid will then be selected by plating onto media containing sucrose. The plasmid encoded sacB gene encodes levan sucrase activity, conversion of sucrose to levan is toxic to gram negative organisms. The second recombination step will result in clones which have either reverted to their pre transformed state or clones containing the integrated promoter antigen fusion, Clones containing the antigen expression cassette would then be identified by PCR. The structure of the construct is depicted in FIG. 5 and SEQ ID NO. 24.

### Example 5

### Construct Containing C. difficile Toxin A CS3 Signal Peptide Fusion

E. coli codon optimized crdA from PPCRscript FAFB as an Apa I/Avr II fragment will be used to construct the promoter CS3 crdA fusion protein. The fragment will then be cloned into the corresponding sites present in vector pMBS CS3 ST. Following confirmation of the structure of this vector the promoter CS3 signal peptide crdA fusion will then be isolated as a Xho I/Avr II fragment and subcloned into a suicide vector based on pCVD442. The suicide vector will have been previously engineered to contain a DNA sequence derived from the intended integration site in the S. typhi genome which will have been modified to include Xho I and Avr II sites at the insertion sites, for example the PCVDaro or pCVDssaV suicide vectors.

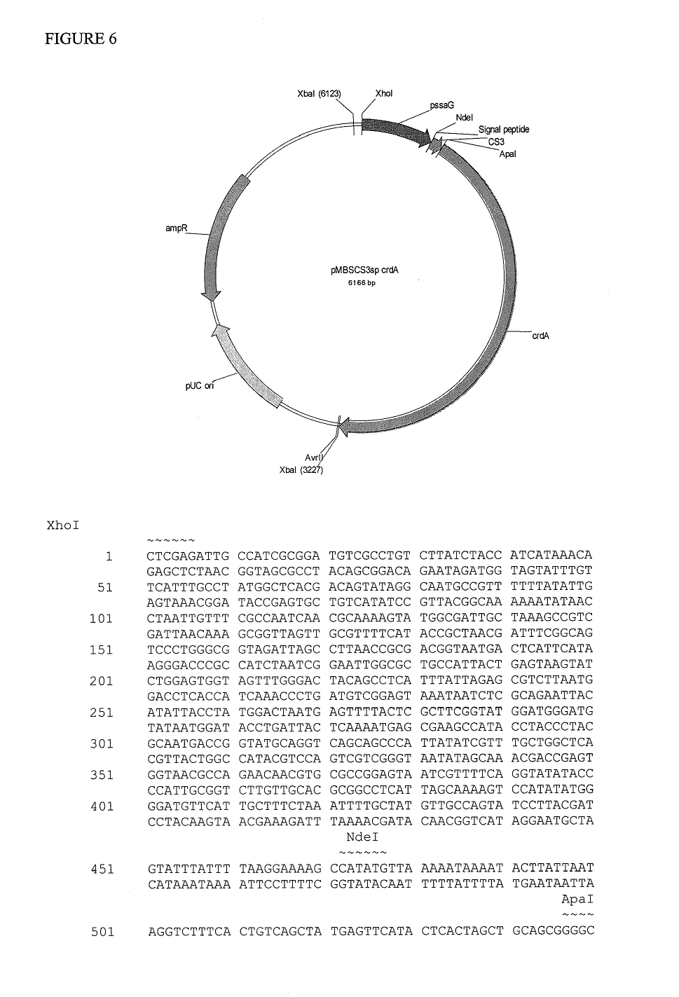

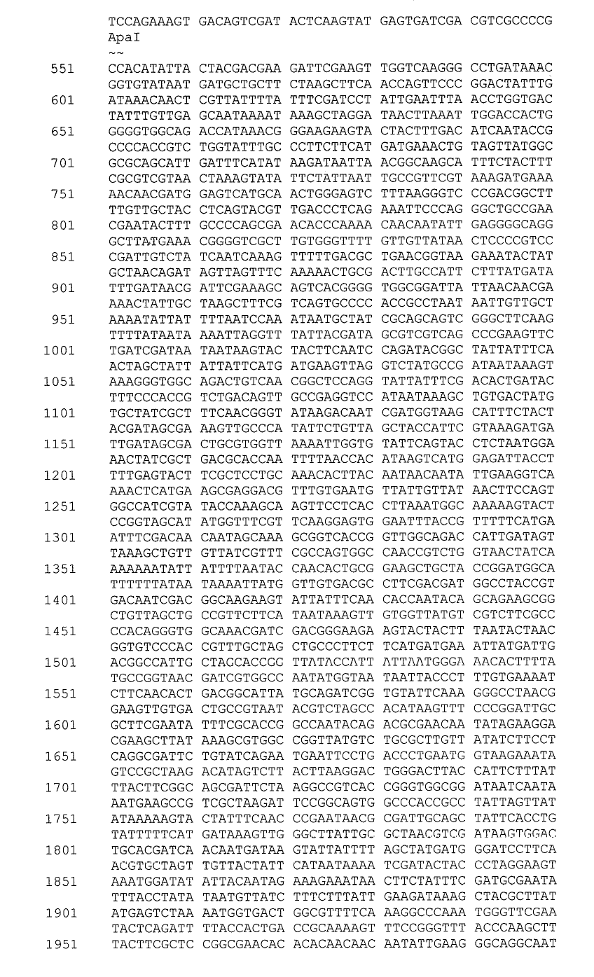

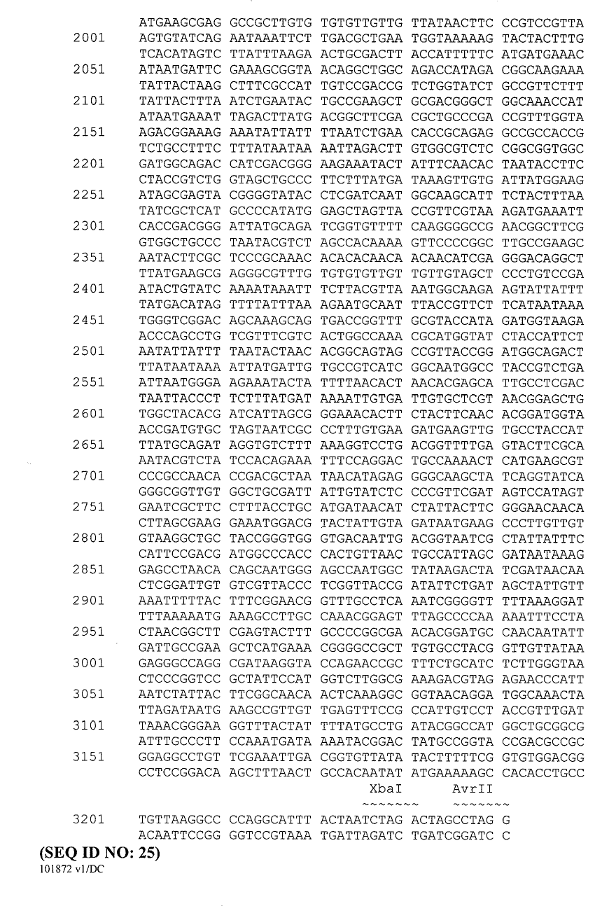

Suicide vectors derived from pCD442 contain the R6k origin of replication which requires the lambda phage pir protein to allow replication in plasmid form. Following transfer of the plasmid into Salmonella by chemical or electro transformation or conjugation from E. coli, the amplicilin marker gene present on the plasmid will only be retained if the plasmid is integrated into the Salmonella chromosome by homologous recombination. Following selection of clones containing the integrated plasmid (merodiploids) these clones will be cultured in the absence of antibiotics to allow a second round of recombination to occur, clones having lost the plasmid will then be selected by plating onto media containing sucrose. The plasmid encoded sacB gene encodes levan sucrase activity, conversion of sucrose to levan is toxic to gram negative organisms. The second recombination step will result in clones which have either reverted to their pre transformed state or clones containing the integrated promoter antigen fusion, Clones containing the antigen expression cassette would then be identified by PCR. The structure of the construct is depicted in FIG. 6 and SEQ ID NO. 25.

### Example 6

### Attenuated Salmonella Strains Expressing Heterologous Antigen Fusions

Attenuated Salmonella strains for use as vaccine candidates can be created by introducing the CS3 fusion constructs described in Examples 1-5 into the attenuated S. typhi ZH9 strain. The ZH9 strain harbors mutations in both the aroC and ssaV genes, each of which is an independently attenuating mutation. See U.S. Pat. No. 6,756,042, which is herein incorporated by reference in its entirety, for a detailed description of the S. typhi ZH9 strain.

As discussed above, any nucleic acid encoding a heterologous antigen may be cloned into either of the two CS3 constructs described in Examples 1-5 to create genetic fusions of either the CS3 export signal sequence and the heterologous antigen or the full length CS3 protein and the heterologous antigen. The CS3 fusions are under the control of a Salmonella ssaG promoter, which is inducible by low pH and low phosphate concentration. The promoter-CS3/antigen fusions can be introduced in the S. typhi ZH9 strain at the site of the aroC deletion mutation by means of a suicide vector. The suicide vector contains the promoter-CS3/antigen fusion flanked by sequences of the aroC deletion sequence found in the S. typhi ZH9 strain.

It is understood that the disclosed invention is not limited to the particular methodology, protocols and reagents described as these may vary. It is also understood that the terminology used herein is for the purposes of describing particular embodiments only and is not intended to limit the scope of the present invention which will be limited only by the appended claims.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the exemplary methods, devices, and materials are as described. All patents, patent applications and other publications cited herein and the materials for which they are cited are specifically incorporated by reference in their entireties.

Those skilled in the art will recognize, or be able to ascertain using no more than routine experimentation, many equivalents to the specific embodiments of the invention described herein. Such equivalents are intended to be encompassed by the following claims.

1. A genetic construct comprising a promoter operably linked to a nucleic acid encoding a fusion protein, wherein said fusion protein comprises an amino acid sequence from E. coli CS3 protein consisting essentially of an export signal fused to at least one heterologous antigen amino acid sequence.

2. The genetic construct of claim 1, wherein the promoter is an inducible promoter.

3. The genetic construct of claim 2, wherein the promoter is a Salmonella ssaG promoter.

4. The genetic construct of claim 1, wherein the amino acid sequence from E. coli CS3 protein is SEQ ID NO: 8.

5. The genetic construct of claim 1, wherein the heterologous antigen amino acid sequence is an amino acid sequence selected from the group consisting of enterotoxigenic E. coli heat stable toxin, enterotoxigenic E. coli heat labile toxin, Chlamydia pmpE, Chlamydia pmpiI, Chlamydia pmpG, Chlamydia htrA, a peptide comprising C. difficile Toxin A C-terminal repeat region, a peptide comprising C. difficile Toxin B C-terminal repeat region, a Hepatitis A antigen, Hepatitis B antigen , Hepatitis C antigen, a Helicobacter antigen, a Herpes Simplex virus antigen, and a human papilloma virus antigen.

6. The genetic construct of claim 1, wherein the fusion protein further comprises a linker amino acid sequence positioned between the CS3 export amino acid sequence and the heterologous antigen amino acid sequence.

7. An attenuated microorganism comprising the genetic construct of claim 1.

8. (canceled)

9. (canceled)

10. The microorganism of claim 7, wherein the microorganism is an attenuated Salmonella.

11. The microorganism of claim 10, wherein the attenuated Salmonella has a deletion or inactivation of a gene involved in the biosynthesis of aromatic compounds.

12. (canceled)

13. The microorganism of claim 10, wherein the attenuated Salmonella has a deletion or inactivation of a gene encoded on the Salmonella pathogenicity island 2 (SPI-2).

14. The microorganism of claim 13, wherein the gene encoded on SPI-2 is ssaV.

15-17. (canceled)

18. A pharmaceutical composition comprising the microorganism of claim 7 and a pharmaceutically acceptable carrier.

19-25. (canceled)

26. A method for inducing an immune response in a subject comprising administering the composition of claim 18 to the subject.

27-32. (canceled)

33. A method for exporting a heterologous antigen from a cell comprising expressing in the cell a genetic construct encoding a fusion protein, wherein said fusion protein comprises an amino acid sequence from E. coli CS3 protein consisting essentially of an export signal fused to at least one heterologous antigen amino acid sequence.

34. The method of claim 33, wherein the fusion protein is expressed from an inducible promoter.

35. The method of claim 34, wherein said promoter is inducible in vivo.

36. The method of claim 34, wherein the inducible promoter is a Salmonella ssaG promoter.

37-45. (canceled)

46. The method of claim 33, wherein the amino acid sequence from E. coli CS3 protein is SEQ ID NO: 8.
